SEQUENCE LISTING (37 sequences)

SEQ ID NO: 1 - DNA, 22 bp, Artificial Sequence
  oligonucleotide
gaagaattgcttccggatgact

SEQ ID NO: 2 - DNA, 17 bp, Artificial Sequence
  oligonucleotide
accaagaggctattcaa

SEQ ID NO: 3 - DNA, 21 bp, Artificial Sequence
  oligonucleotide
  misc_feature (14)..(17): n is a, c, g, or t
cttcctccactgcnnnncaga

SEQ ID NO: 4 - DNA, 21 bp, Artificial Sequence
  oligonucleotide
  misc_feature (14)..(17): n is a, c, g, or t
cttcctccactgcnnnncaga

SEQ ID NO: 5 - DNA, 21 bp, Artificial Sequence
  oligonucleotide
  misc_feature (14)..(17): n is deoxyinosine
cttcctccactgcnnnncaga

SEQ ID NO: 6 - DNA, 21 bp, Artificial Sequence
  oligonucleotide
  misc_feature (14)..(17): n is 5-nitroindole
cttcctccactgcnnnncaga

SEQ ID NO: 7 - DNA, 22 bp, Artificial Sequence
  oligonucleotide
gaagaattgcttccggatgact

SEQ ID NO: 8 - DNA, 19 bp, Artificial Sequence
  oligonucleotide
catttttggtttgggcttc

SEQ ID NO: 9 - DNA, 172 bp, Artificial Sequence
  oligonucleotide
  misc_feature (16)..(16): W is A or T
  misc_feature (17)..(17): R is A or G
gacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctgtttggcagtggaggaagtctctttaag

SEQ ID NO: 10 - DNA, 321 bp, Artificial Sequence
  oligonucleotide
gacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgt

SEQ ID NO: 11 - DNA, 325 bp, Artificial Sequence
  oligonucleotide
  misc_feature (146)..(149): n is a, c, g, or t
gacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctgnnnngcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgt

SEQ ID NO: 12 - DNA, 21 bp, Artificial Sequence
  oligonucleotide
  misc_feature (5)..(8): n is a, c, g, or t
tctgnnnngcagtggaggaag

SEQ ID NO: 13 - DNA, 21 bp, Artificial Sequence
  oligonucleotide
  misc_feature (5)..(5): Y is C or T
  misc_feature (6)..(6): W is A or T
tctgywtggcagtggaggaag

SEQ ID NO: 14 - DNA, 21 bp, Artificial Sequence
  oligonucleotide
  misc_feature (16)..(16): W is A or T
  misc_feature (17)..(17): R is A or G
cttcctccactgccawrcaga

SEQ ID NO: 15 - DNA, 885 bp, Homo sapiens
atggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaatctatacgagatactccagccaaaaatgcacaaaagtcaaatcagaatggaaaagactcaaaaccatcatcaacaccaagatcaaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaa

SEQ ID NO: 16 - DNA, 21 bp, Artificial Sequence
  oligonucleotide
cttcctccactgccagacaga

SEQ ID NO: 17 - DNA, 19 bp, Artificial Sequence
  oligonucleotide
cctccactgccatgcagag

SEQ ID NO: 18 - DNA, 18 bp, Artificial Sequence
  oligonucleotide
cctccactgccaggcaga

SEQ ID NO: 19 - DNA, 19 bp, Artificial Sequence
  oligonucleotide
cctccactgccaagcagag

SEQ ID NO: 20 - DNA, 21 bp, Artificial Sequence
  oligonucleotide
cttcctccactgccatacaga

SEQ ID NO: 21 - DNA, 19 bp, Artificial Sequence
  oligonucleotide
ctccactgcccgacagaga

SEQ ID NO: 22 - DNA, 23 bp, Artificial Sequence
  oligonucleotide
cttcctccactgccttacagaga

SEQ ID NO: 23 - DNA, 17 bp, Artificial Sequence
  oligonucleotide
cctccactgccacgcag

SEQ ID NO: 24 - DNA, 19 bp, Artificial Sequence
  oligonucleotide
tcctccactgccaaacaga

SEQ ID NO: 25 - DNA, 19 bp, Artificial Sequence
  oligonucleotide
tcctccactgctttgcaga

SEQ ID NO: 26 - DNA, 22 bp, Artificial Sequence
  oligonucleotide
cttcctccactgccctacagag

SEQ ID NO: 27 - DNA, 18 bp, Artificial Sequence
  oligonucleotide
cctccactgccgagcaga

SEQ ID NO: 28 - DNA, 19 bp, Artificial Sequence
  oligonucleotide
tcctccactgaacggcaga

SEQ ID NO: 29 - DNA, 21 bp, Artificial Sequence
  oligonucleotide
cttcctccactgcctggcaga

SEQ ID NO: 30 - DNA, 21 bp, Artificial Sequence
  oligonucleotide
cttcctccactgcccggcaga

SEQ ID NO: 31 - DNA, 172 bp, Artificial Sequence
  oligonucleotide
gacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctgccgttcagtggaggaagtctctttaag

SEQ ID NO: 32 - DNA, 172 bp, Artificial Sequence
  oligonucleotide
gacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctgcaaagcagtggaggaagtctctttaag

SEQ ID NO: 33 - DNA, 172 bp, Artificial Sequence
  oligonucleotide
gacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctgcgtggcagtggaggaagtctctttaag

SEQ ID NO: 34 - DNA, 172 bp, Artificial Sequence
  oligonucleotide
gacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctgctcggcagtggaggaagtctctttaag

SEQ ID NO: 35 - DNA, 172 bp, Artificial Sequence
  oligonucleotide
gacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctgtagggcagtggaggaagtctctttaag

SEQ ID NO: 36 - DNA, 172 bp, Artificial Sequence
  oligonucleotide
gacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctgtatggcagtggaggaagtctctttaag

SEQ ID NO: 37 - DNA, 172 bp, Artificial Sequence
  oligonucleotide
gacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctgtcgggcagtggaggaagtctctttaag

(19) United States
(12) Patent Application Publication
(10) Pub. No.: US 2019/0144950 A1
(43) Pub. Date: May 16, 2019

(54) METHODS OF DETECTING A MUTATED GENE BY MULTIPLEX DIGITAL PCR

(71) Applicant: CORNELL UNIVERSITY, Ithaca, NY (US)

(72) Inventor: Duane HASSANE, New York, NY (US)

(21) Appl. No.: 16/301,202

(22) PCT Filed: May 18, 2017
(86) PCT No.: PCT/US17/33314

§ 371 Date: Nov. 13, 2018

Related U.S. Application Data
(60) Provisional application No. 62/338,058, filed on May 18, 2016.

(51) Int. Cl. C12Q 1/6886

(52) U.S. Cl. CPC C12Q 1/6886; C12Q 2600/156

(57) ABSTRACT

The present disclosure is directed to digital PCR (dPCR)-based methods and kits for detecting and quantifying mutant nucleic acids (e.g., transcripts) of a gene containing insertions at specific locations. In some embodiments, the method permits sensitive detection and quantitation of mutant NPM1 nucleic acids comprising insertion mutations (NPM1mut subtypes).

### CROSS REFERENCE TO RELATED APPLICATION

This application claims the benefit of priority from U.S. Provisional Application No. 62/338,058, filed May 18, 2016, the entire contents of which are incorporated herein by reference.

### INCORPORATION BY REFERENCE OF SEQUENCE LISTING

The Sequence Listing in an ASCII text file, named as 33571PCT.7375-02-PC.Seq_ST25.txt of 11 KB, created on May 18, 2017, and submitted to the United States Patent and Trademark Office via EFS-Web, is incorporated herein by reference.

### BACKGROUND

Acute myeloid leukemia (AML) is a fatal disease with dismal outcomes. Despite initial remissions, most patients relapse and ultimately succumb to their disease. The presence of minimal residual disease (MRD) in patients using molecular and immunological criteria has been shown to be informative of outcome (Grimwade et al., J. of Clin. One., (2009), 27 (22): 3650-58; Hourigan and Karp, Nature Reviews. Clinical Oncology, (2013), 10 (8): 460-71; Perea et al., Leukemia, (2006), 20 (1): 87-94; Paietta, (2015), Best Practice & Research Clinical Haematology, 28 (2-3): 98-105). Immunological methods of MRD detection are effective but require multi-antibody panels as well as the flow cytometric identification of rare cell populations. Molecular methods of MRD evaluation represent a complementary or alternative approach easily accomplished by PCR in about 30% of AML patients with abnormal cytogenetics testing for lesions such as inv(16), AML1-ETO, and PML-RARA. About half of AMLs are cytogenetically normal (CN-AML) lacking MRD markers identifiable by standard cytogenetics or FISH. Sequencing analyses of these CN-AML have shown that 40-50% have NPM1 mutations. Thus, MRD testing for NPM1-mutated AML by real time quantitative PCR (RQ-PCR) with allele-specific oligonucleotide primers has become a well-established option for these patients practiced in numerous studies across multiple centers (Gorello et al., Leukemia, (2006), 20 (6): 1103-8; Grimwade et al., J. of Clin. Onc., (2009), 27 (22): 3650-58). Approximately 95% of NPM1 mutations consist of 4 nucleotide (nt) insertions in exon 12 at the 863 position, the most common of which is “type A” (c.860_863dupTCTG), found in approximately 75% of NPM1 mutated AML patients. (Schnittger et al., Blood, (2005), 106 (12): 3733-39; Thiede et al., Blood, (2006), 107 (10): 4011-20; Verhaak et al., Blood, (2005), 106 (12): 3747-54; Perea et al., Leukemia, (2006), 20 (1): 87-94; Andersen et al., Leukemia, (2008), 22 (5): 951-55; Falini et al., Haematologica, (2007), doi: 10.3324/haematol.11007). The remaining patients often have rarer subtypes with differing and sometimes highly patient-specific insertion sequences with over 50 such polymorphisms being reported (Bacher et al., British Journal of Haematology, (2004), 167 (5): 710-14) and at least hundreds being theoretically possible. Unlike cytogenetic markers, MRD testing in patients with NPM1-mutated AML currently requires prior DNA sequencing to identify the specific insertion sequence which is then paired to a NPM1 allele-specific PCR test. Quantitative MRD testing thus requires maintenance of assays and plasmid standards for each polymorphism with commercial standards being widely available for only the top 3 NPM1 mutations (types A, B and D). Although RQ-PCR assays have been directly adapted to digital PCR (dPCR) to circumvent plasmid standards (Bacher et al., British Journal of Haematology, (2004), 167 (5): 710-14), custom primers and/or probes are still required. Moreover, in many patients, NPM1-mutated diagnosis is made without sequencing using inexpensive capillary electrophoresis (Szankasi et al., JMD, (2008), 10 (3): 236-41), which precludes reliable MRD testing with any current approach.

Therefore, a substantial number of patients with NPM1-mutated AML are ineligible for MRD assessment because they lack NPM1 sequence information and/or the availability of an appropriate test. Finally, despite reports of overall NPM1 stability (Palmisano et al., Haematologica, (2007), 92 (9), 1268-69; Kristensen et al., European Journal of Haematology, (2011), 87 (5): 400-408; Meloni et al., Haematologica, (2009) 94 (2): 298-300), the reliability of NPM1 mutations as an MRD marker using current detection approaches is further complicated by reported instances of intra-patient NPM1 heterogeneity (Salipante et al., Modern Pathology, (2014) 27, 1438-1446) and type-switching (Webersinke et al., Blood Cancer Journal, (2014), 4 (6)), both of which can produce misdiagnosed MRD status in isolated cases.

Clinical decisions in AML are informed currently by individual patient characteristics including age, clinical history, cytogenetic information, and recently established molecular criteria according the WHO classification. Despite these advances, outcomes have remained largely unchanged for the past 40 years and thus there is an urgent need for improved therapeutics and treatment algorithms. Recent studies suggest that assessment of mutant NPM1 transcript copies as a MRD marker is improves risk assessment relative to established cytogenetic and molecular criteria alone (Perea et al., Leukemia. (2006), 20 (1): 87-94; Kern et al. 2008, Cancer. doi: 10.1002/cncr.23128; Paietta, Best Practice & Research Clinical Haematology, (2015), 28 (2-3): 98-105; Hourigan and Karp, Nature Reviews Clinical Oncology, (2015), 10 (8): 460-71; Ivey et al., The New England Journal of Medicine, (2016), 374 (5)). Indeed, a recent prospective study of 223 patients revealed subgroups of NPM1-mutated patients with improved survival when NPM1 negative by PCR despite having prognostically adverse co-occurring mutations such as FLT3-ITD and/or DNMT3A (Ivey et al., The New England Journal of Medicine, (2016), 374 (5)). MRD monitoring through assessment of residual mutated NPM1 transcript copies is thus likely to become a more significant prognostic tool used by AML oncologists.

Despite the efficacy of current NPM1 tests, a substantial number of patients with NPM1-mutated AML are currently ineligible for quantitative MRD monitoring either because their NPM1 mutation is rare or because their insertion sequence is unknown since detection was performed using capillary electrophoresis. It is possible to employ RQ-PCR difference-in-cycle thresholds (Grimwade et al., J. of Clin. Onc., (2009), 27 (22): 3650-58) as an alternative for rare NPM1 mutations, but this approach is sensitive to input RNA making cross-study comparisons challenging especially when patient materials are limiting and PCR efficiency can vary without consistent reporting. Developing many patient-tailored tests is also a possibility but may pose challenges as NPM1 mutation testing transitions into tightly regulated clinical settings where each type-specific test and its corresponding quantitative standard requiring rigorous quality control and certification. Finally, reports suggesting intra-patient NPM1 heterogeneity and type-switching indicate the possibility of MRD underestimation or misdiagnosis in at least some subset of patients.

Minimal Residual Disease (MRD) testing would be greatly improved by a more robust assay that is usable on more patients, that simplifies the testing process for practitioners, and that better addresses the possibility of intra-patient heterogeneity or clonal evolution. Digital PCR (dPCR) is an emergent technology with most applications thus far being direct adaptations of a Real-time Quantitative PCT (RQ-PCR) counterpart (Baker, Nature Methods, (2012) 9 (6) 541-44).

### SUMMARY OF THE DISCLOSURE

In one aspect, this disclosure provides a method of detecting mutant nucleic acid molecules of a gene in a sample, wherein the mutant nucleic acid molecules of the gene comprise an insertion of at least one nucleotide at a specific site in the gene.

In some embodiments, the method includes performing digital PCR on a sample suspected of containing mutant nucleic acid molecules of a gene using a first primer set, a second primer, and a first probe. In these embodiments, (i) the first primer set comprises multiple primers, wherein the multiple primers anneal specifically to mutant nucleic acid molecules of the gene that comprise an insertion of at least one nucleotide at a specific site in the gene, and the multiple primers are degenerate in at least one nucleotide position corresponding to a position of an inserted nucleotide in the mutant nucleic acid molecules; (ii) the second primer anneals specifically to a sequence in said gene to permit generation of amplicons when used with the first primer set in the digital PCR, and (iii) the first probe hybridizes to a sequence in said gene located between the site of insertion and the sequence to which the second primer hybridizes.

In some embodiments, the multiple primers in the first primer set are degenerate in at least 2-4 nucleotide positions corresponding to positions of inserted nucleotides in the mutant nucleic acid molecules.

In some embodiments, the multiple primers in the first primer set comprises nucleotides at two to four adjacent positions corresponding to positions of inserted nucleotides in the mutant nucleic acid molecules, wherein the nucleotide at each of the at least two to four positions is independently A, C, G, T, dI, 5NI or another modified nucleotide, and wherein the multiple primers are degenerate in at least one of the two-four positions.

In some embodiments, the multiple primers in the first primer set comprises nucleotides at four adjacent positions (NNNN) corresponding to positions of inserted nucleotides in the mutant nucleic acid molecules, wherein N at each position is independently A, C, G, T, dI, 5NI or another modified nucleotide, and wherein the multiple primers are degenerate in at least two of the four positions. In certain embodiments, the multiple primers are degenerate at all four positions.

In some embodiments, degeneracy at a nucleotide position in the first primer set is achieved by using a mixture of selected nucleotides. In some embodiments, the mixture of selected nucleotides is a mixture of A, C, G, T, dI, and 5NI, a mixture of A, C, G, and T, a mixture of C and T, a mixture of A and T, or a mixture of A and G.

In some embodiments, the selected nucleotides in the mixture are at a predetermined ratio relative to each other. In some embodiments, the amounts of the selected nucleotides in the mixture are equal relative to each other; and in other embodiments, the amounts of the selected nucleotides in the mixture are not equal relative to each other, and are predetermined to favor detection of common insertion mutations.

In some embodiments, the first primer set and the second primer are designed to provide amplicons of about 75-200 base pairs.

In some embodiments, the gene is NPM1. In some embodiments, the insertion is selected from the group consisting of insertions between positions 859 and 860, positions 860 and 861, positions 861 and 862, positions 862 and 863, positions 863 and 864, positions 864 and 865, positions 865 and 866, positions 866 and 867, and positions 867 and 868 of NPM1, with the position numbering based on the wild-type NPM1 coding sequence as set forth in SEQ ID NO: 15. In some embodiments, the insertion is in exon 11 or exon 12 of NPM1. In some embodiments, the insertion is between positions 863-864 of NPM1.

In some embodiments, the multiple primers in the first primer set are degenerate at two to four positions corresponding to positions of inserted nucleotides in mutant NPM1. In specific embodiments, the multiple primers in the first primer set are degenerate at four positions corresponding to positions of inserted nucleotides in mutant NPM1.

In some embodiments, the multiple primers in the first primer set comprises nucleotides at two to four adjacent positions corresponding to positions of inserted nucleotides in mutant NPM1 molecules, wherein the nucleotide at each of the at least two to four positions is independently A, C, G, T, dI, 5NI or another modified nucleotide, and wherein the multiple primers are degenerate in at least one of the two to four positions.

In some embodiments, the multiple primers in the first primer set comprises nucleotides at four adjacent positions (5′ NNNN 3′), wherein the multiple primers are degenerate in at least two of the four positions, and wherein N at each position is independently A, C, G<T, dI, 5NI, or another modified nucleotide. In some embodiments, the multiple primers are degenerate at all four positions. In some embodiments, at least one N is fixed to a nucleotide selected from the group consisting of A, T, G or C. In some embodiments, the first N position is Y, the second N position is W, the third N position is T and the fourth N position is G, wherein Y is C or T and W is A or T.

In some embodiments, degeneracy is skewed such that the first N is 20% A, 45% C, 10% G, 25% T, the second N is 60% A, 15% C, 10% G, 15% T and the third N is 15% A, 15% C, 60% G, 10% T. In some embodiments, the skewing of degeneracy can be adjusted to favor detection of common insertion mutations.

In some embodiments, the multiple primers in the first primer set are forward primers and comprise the sequence, 5′-TCTGNNNNGCAGTGGAGGAAG-3′ (SEQ ID NO: 12), wherein N at each position is independently A, C, T, or G and at least 2 of the 4 positions or all 4 positions denoted by N are degenerate. In other embodiments, the multiple primers in the first primer set are reverse primers and comprise the sequence, 5′-CTTCCTCCACTGCNNNNCAGA-3′ (SEQ ID NO: 4), wherein N at each position is independently A, C, T, or G and at least 2 of the 4 positions or all 4 positions denoted by N are degenerate.

In some embodiments, the multiple primers in the first primer set are forward primers represented by the sequence 5′-TCTGYWTGGCAGTGGAGGAAG-3′ (SEQ ID NO: 13), wherein Y is C or T and W is A or T. In other embodiments, the multiple primers in the first primer set are reverse primers represented by the sequence 5′-CTTCCTCCACTGCCAWRCAGA-3′ (SEQ ID NO: 14), wherein W is A or T and R is A or G.

In some embodiments, the first primer set and the second primer are designed to provide amplicons of about 75-200 base pairs. For detecting insertion mutations in exon 12, in specific embodiments, the second primer is a forward primer and hybridizes to a sequence in exon 9, 10, 11 or a combination of exons 9, 10 and 11 of NPM1, and in other specific embodiments, the second primer is a reverse primer and hybridizes to a sequence in exon 12 of NPM1. In a specific embodiment, the second primer comprises the sequence, 5′-GAAGAATTGCTTCCGGATGACT-3′ (SEQ ID NO: 1).

In some embodiments, the first probe comprises a fluorescent label and a quencher. In specific embodiments, the first probe comprises the sequence, 5′-ACCAAGAGGCTATTCAA-3′ (SEQ ID NO: 2).

In some embodiments, the mutant nucleic acids are RNA, cDNA or DNA molecules.

In further embodiments, the method further includes quantifying the mutant nucleic acids of the NPM1 gene in a sample.

In some embodiments, the quantification can include detecting and quantifying nucleic acids of a second gene in the sample and generating a ratio of mutant nucleic acids of the NPM1 gene to the nucleic acids of the second gene. In specific embodiments, the second gene can be selected from ABL1, wild-type NPM1, or total NPM1 (mutant and wild type NPM1 combined).

In some embodiments, a second probe is included in the assay that specifically hybridizes to ABL1 and comprises a second fluorescent label different from the fluorescent label of the first probe. The second probe can include the same or different quencher from the quencher used in the first probe.

In further embodiments, the number of any mutated NPM1 transcript greater than or equal to a detection limit is indicative of cancer or residual cancer cells. In some embodiments, the cancer is a blood cancer. In specific embodiments, the blood cancer is selected from the group consisting of non-Hodgkin's lymphoma, acute promyelocytic leukemia, myelodysplastic syndrome, acute lymphocytic leukemia and acute myelogenous leukemia.

In some embodiments, the sample used in the assay disclosed herein is sample of a cancer patient who has undergone cancer therapy.

In a further aspect, this disclosure provides a kit comprising a first primer set useful for detection of mutant nucleic acids in the NPM1 gene in a dPCR based assay disclosed herein.

In some embodiments, the first primer set in the kit includes multiple primers that specifically anneal to a sequence in mutant NPM1 nucleic acid molecules encompassing an insertion at a specific site (e.g., between positions 859 and 860, positions 860 and 861, positions 861 and 862, positions 862 and 863, positions 863 and 864, positions 864 and 865, positions 865 and 866, positions 866 and 867, and positions 867 and 868, particularly between positions 863 and 864), wherein the multiple primers within the first primer set are degenerate in at least one position corresponding to a position of an inserted nucleotide at the specific site.

In some embodiments, the multiple primers in the first primer set comprises nucleotides at two to four adjacent positions corresponding to positions of inserted nucleotides in mutant NPM1 molecules, wherein the nucleotide at each of the at least two to four positions is independently A, C, G, T, dI, 5NI or another modified nucleotide, and wherein the multiple primers are degenerate in at least one of the two to four positions.

In some embodiments, the multiple primers within the first primer set comprise four adjacent nucleotides (5′ NNNN 3′) at positions corresponding to inserted nucleotides in mutant NPM1 molecules, wherein N at each position is independently A, C, G, T, dI, 5NI or another modified nucleotide, and wherein at least one or two of the four positions denoted by N are degenerate. In some embodiments, at least one N is fixed to a nucleotide selected from the group consisting of A, T, G or C. In some embodiments, the first N is Y, the second N is W, the third N is T and the fourth N is G, wherein Y is C or T and W is A or T. In some embodiments, all four positions are degenerate.

In specific embodiments, the multiple primers comprises the sequence, 5′-CTTCCTCCACTGCNNNNCAGA-3′ (SEQ ID NO: 4), wherein N at each position is independently A, C, T, or G, and wherein at least one or two of the four positions denoted by N are degenerate. In some embodiments, the multiple primers in the first primer set are forward primers represented by the sequence to 5′-TCTGYWTGGCAGTGGAGGAAG-3′ (SEQ ID NO: 13). In other embodiments, the multiple primers in the first primer set are reverse primers represented by the sequence to 5′-CTTCCTCCACTGCCAWRCAGA-3′ (SEQ ID NO: 14), wherein W is A or T and R is A or G.

In some embodiments, the kit further includes a second primer that hybridizes to a sequence in NPM1, wherein the first primer set and the second primer are designed to provide amplicons of about 75-200 base pairs in length.

In some embodiments, the kit also includes a probe that hybridizes to a sequence in NPM1 located between the site of insertion in NPM1 and the sequence in NPM1 to which the second primer hybridizes. In some embodiments, the probe is labeled with a flurophore and a quencher.

### BRIEF DESCRIPTION OF THE DRAWINGS

FIG. 1A-1D. Cross-detection between NPM1 assays motivates design of a dPCR-based multiplex NPM1 assay. (A) RQ-PCR amplification curves indicating normalized reporter value (ARn) as a function of PCR cycle. The EAC NPM1 type A assay is able to amplify 2,000 copies of Ipsogen reference plasmids for NPM1 type A (diamonds) and B mutations (triangles) but not wild-type NPM1 (circles). (B) Percent cross-detection between combinations of type-specific assays and NPM1 mutations alongside wild-type NPM1. (C) Histograms indicate the distribution of positive droplets of type-specific, multiplex, and deoxyinosine-based NPM1 mutation assays in NPM1 type A patient sample (NPM1mut AML; Type A) alongside NPM1 negative mononuclear cells (negative). The vertical axis indicates log₁₀(absolute counts) with the horizontal axis indicating droplet signal intensity. The vertical dashed line indicates the positive threshold. CV of signal intensities for the positive population is indicated. (D) Schematic of an embodiment of the massively multiplex assay indicating relative positions of the common forward primer, multiplex reverse primer, and common probe. Productive amplification occurs with NPM1mut templates (top) but not NPM1 wild-type templates (bottom).

FIG. 2A-2E. Performance of multiplex NPM1 MRD assay on spike-in dilution series. (A) Correlation of RQ-PCR-based EAC type A-specific assay (vertical axis) to dPCR-based adaptation of the EAC type A-specific assay (horizontal axis) when using cDNA derived from either undiluted OCI-AML3 or OCI-AML3 spiked into GM12878 at 1:1000, 1:10,000, 1:50,000 cells or GM12878 alone. Axes indicate NPM1mut/10⁴ ABL1. (B) Correlation of the massively multiplex NPM1 assay (vertical axis) to dPCR-based adaptation of the EAC type A-specific assay (horizontal axis) on the same dilution series as panel A. Axes indicate NPM1mut/10⁴ ABL1. (C) Dotplot of dPCR data for the multiplex assay (top row) and type A-specific EAC assay adapted to dPCR (bottom row) on the same spike-in dilution series as panels A and B. Dilutions are indicated. The vertical axis indicates the signal intensity of droplets in the ABL1 channel (VIC) and the horizontal axis indicates positive droplets in the NPM1 mutant channel (FAM). NPM1mut/10⁴ ABL1 copies are indicated in the upper right hand corner of each scatter plot. Positive droplets are emphasized in blue. (D) Correlation of the massively multiplex NPM1 assay (vertical axis) to dPCR-based adaptation of the EAC type A-specific assay (horizontal axis) on cDNA from NPM1 mutated AML (type A) either undiluted or diluted into cDNA from healthy cord blood at 1:1000, 1:10,000 (w/w) alongside cord blood cDNA alone. Axes indicate NPM1mut/10⁴ ABL1. (E) Same as panel D except using a type DD1-specific dPCR on cDNA from a rare NPM1 mutated AML (type DD1). Lin's concordance correlation coefficient is indicated (ρ_{c}).

FIG. 3A-3F. Multiplex NPM1 assay accurately detects rare NPM1 mutations types. (A) Schematic representation of synthetic NPM1mut target consisting of a pool of degenerate NPM1mut insertion sequences. Amplification is performed using common forward primer with either multiplex reverse primer pool (mutant-specific) or the universal reverse primer (amplifies both mutant and wild-type). Common probe is used for all reactions. (B) Histogram indicating counts and distribution of fluorescence signal intensity of NPM1 positive and negative droplets. (C) Plasmids harboring NPM1 type A (top row), type B (middle row), or a 50:50 mixture of type A and B (bottom row) mutations were spiked into GM12878 background cDNA. A target of 350 NPM1mut/10⁴ ABL1 copies was used in all cases and the expected content indicated graphically by the barplot on each row. Detection was attempted by dPCR using the specific assays for the type A (left column) or type B (middle colum) mutation alongside the massively multiplex assay (right column). The vertical axis of each dotplot indicates the signal intensity of ABL1 positive droplets and the horizontal axis indicates NPM1mut positive droplets. NPM1mut/10⁴ ABL1 ratios are indicated in the upper right corner. (D) Synthetic template for NPM1 c.863_864insTATG is spiked into GM12878 cDNA targeting approximately 1,000 NPM1mut/10⁴ ABL1 copies and either left undiluted (1×) or diluted 4-fold (0.25×) or 10-fold (0.1×) alongside negative control cell line GM12878. The axes indicate NPM1mut/10⁴ ABL1 as determined by the NPM1 multiplex assay (vertical axis) vs. type-specific assay (horizontal axis). (E) Scatterplot of dPCR data for the multiplex assay (left column) and c.863_864insTATG-specific assay adapted to dPCR (right column) for the dilution series from panel A with dilutions 1×, 0.25×, and 0.1× shown. (F) Correlation between NPM1mut/10⁴ ABL1 ratios for a different synthetic rare NPM1 insertion mutations spiked into GM12878 cDNA targeting an approximately 1,000 NPM1mut/10⁴ ABL1 starting ratio. Undiluted spike-in mixture (1×) is then compared to 4-fold (0.25×) and 10-fold (0.1×). The position 863 insertion sequence is indicated over each plot except for delGinsCCGTT, which is NPM1 c.864_865delGinsCCGTT. The axes indicate NPM1mut/10⁴ ABL1 ratios for the multiplex assay (vertical axis) vs. type-specific assays (horizontal axis). Lin's concordance correlation coefficient is indicated (ρ_{c}).

FIG. 4A-4E. Monitoring of MRD in serial AML cases. (A) Patient 1 (NPM1 type B mutated) NPMmut/10⁴ ABL1 as a function of time. The vertical axis indicates NPM1mut/10⁴ ABL1, the horizontal axis indicates time of since initial monitoring in days. Diamond points indicate results with the type B-specific assay. Circular points indicate results with the NPM1 multiplex assay. (B) Patient 2 (NPM1 type D mutated) NPM1mut/10⁴ ABL1 as a function of time. The vertical axis indicates NPM1mut/10⁴ ABL1, the horizontal axis indicates time of since initial monitoring in days. Diamond points indicate results with the type D-specific assay. Circular points indicate results with the NPM1 multiplex assay. (C) Patient 3 (NPM1 c.865_866ins(CAGC)) NPM1mut/10⁴ ABL1 as a function of time. The vertical axis indicates NPM1mut/10⁴ ABL1, the horizontal axis indicates time of since initial monitoring in days. Diamond points indicate results with the CAGC type-specific assay. Circular points indicate results with the NPM1 multiplex assay. PB samples are shown on the bottom panel and BM samples on the middle panel. (D) The insertion sequence identified by deep targeted mRNA-seq for Patient 2 above the alignment of reads supporting the NPM1 type B insertion. (E) Reads supporting IDH1 (p.ArgR132His) mutation identified by deep targeted mRNA-seq for Patient 2.

FIG. 5A-5C. Performance of multiplex NPM1 MRD assay on spike-in dilution series on alternative dPCR platform and with higher cDNA input. (A) Correlation of RQ-PCR-based EAC type A-specific assay (vertical axis) to dPCR-based adaptation of the EAC type A-specific assay on the BioRad QX200 (horizontal axis) when using cDNA derived from either undiluted OCI-AML3 or OCI-AML3 spiked into GM12878 at 1:1000, 1:10,000, 1:50,000 cells or GM12878 alone. Axes indicate NPM1mut/10⁴ ABL1. R²>0.99; linear regression (p<0.0001). (B) Correlation of the massively multiplex NPM1 assay on RainDance RainDrops (vertical axis) to dPCR-based adaptation of the EAC type A-specific assay on BioRad QX200 (horizontal axis) on the same dilution series as panel A. Axes indicate NPM1mut/10⁴ ABL1. R²>0.99; linear regression (p<0.0001). (C) Scatterplot of dPCR data for the multiplex assay (top row) and type A-specific EAC assay adapted to dPCR (bottom row) on the same spike-in dilution series as panels A and B but with 10× as much input to ascertain improved sensitivity. Dilutions are indicated. The vertical axis indicates the signal intensity of droplets in the ABL1 channel (VIC) and the horizontal axis indicates positive droplets in the NPM1 mutant channel (FAM). NPM1mut/10⁴ ABL1 copies are indicated in the upper right hand corner of each scatter plot. BioRad QX200 could not be tested in panel C because the number of ABL1 copies saturated the instruments so that it was no longer quantitative with respect to ABL1.

FIG. 6A-6C. The type-specific and massively multiplex assay agree in rare NPM1 mutations observed in patients. cDNA from NPM1-mutated AML was diluted into cDNA from healthy cord blood at 1:1,000, 1:10,000 (w/w) alongside cord blood (CB) controls. Detection was performed with either the multiplex assay or the corresponding type-specific assay. Concordance of the massively multiplex NPM1 assay (vertical axis) to type-specific assays (horizontal axis) is shown for (A) NPM1 c.863_864insTATG, (B) NPM1 c.865_866insCAGC, and (C) NPM1 c.863_864insGCGG. The axes indicate detected NPM1mut/10⁴ ABL1 ratios. Lin's concordance correlation coefficient (ρ_{c}) is indicated.

### DETAILED DESCRIPTION

In the following description, reference is made to the accompanying drawings that form a part hereof, and in which is shown by way of illustration specific embodiments which may be practiced. These embodiments are described in detail to enable those skilled in the art to practice the invention, and it is to be understood that other embodiments may be utilized and that logical changes may be made without departing from the scope of the present invention. The following description of example embodiments is, therefore, not to be taken in a limited sense, and the scope of the present invention is defined by the appended claims.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

### Definitions

Unless defined otherwise, all technical and scientific terms used herein have the same meaning as commonly understood by one of ordinary skill in the art to which this invention belongs.

As used herein, the term “about” refers to an approximately +/−10% variation from a given value.

The term “amplification” or “amplify” as used herein includes methods for copying a target nucleic acid, thereby increasing the number of copies of a selected nucleic acid sequence. Amplification may be exponential or linear. A target nucleic acid may be either DNA or RNA. The regions or sequences of a target nucleic acid amplified in this manner form an “amplicon” or “amplification product”. While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols (1990), Innis et al., Eds., Academic Press, San Diego, Calif., pp 13-20; Wharam, et al., Nucleic Acids Res. (2001), June 1; 29(11):E54-E54; Hafner, et al., Biotechniques (2001), 4:852-6, 858, 860.

The term “antisense strand” refers to a nucleotide sequence that is complementary to a DNA sequence. The term “sense strand” refers to a nucleotide sequence that corresponds to a DNA sequence and thus is complementary to the antisense strand.

The term “cancer therapy” refers to treatment of cancer with various methods including, but not limited to, formulations of chemicals (chemotherapy), or combined modality therapies that may include chemotherapy, radiation therapy, and/or surgery.

As used herein, the term “detection limit” (also known as “limit of detection (LOD)”) refers to the lowest level or amount of an analyte, such as a nucleic acid, that can be detected and quantified. Limits of detection can be represented as molar values (e.g., 2.0 nM limit of detection), as gram measured values (e.g., 2.0 microgram limit of detection under, for example, specified reaction conditions), copy number (e.g., 1×10⁵ copy number limit of detection), copy number over a normalizing copy number (e.g. 1.0 target gene A/10⁴ normalizer gene B) or other representations known in the art. For instance, in the present disclosure, the limit of detection (LOD) for the multiplex and type A-specific assays under dPCR conditions are estimated at 2.4 and 1.0 NPM1mut/10⁴ ABL1, respectively.

The term “diagnose” as used herein, refers to an intervention performed with the intention of determining the presence of a disease or condition, as well as the likelihood of a subject having that disease or condition, as well as a subject's response to an intervention to treat or prevent of the disease or condition, as well as evaluation of minimal residual disease, as well as determination of appropriate treatment for a given subject (often called “theranostic”), as well as evaluating whether a subject should be included in a clinical trial. Those in need of diagnosis include those already having the disease, disorder or condition, those suspected of having the disease, disorder, or condition, those prone to, or at risk of developing, the disease, disorder or condition and those in whom the disease, disorder or condition is to be prevented.

The term “DNA,” as used herein, refers to a nucleic acid molecule of one or more nucleotides in length, wherein the nucleotide(s) are nucleotides. By “nucleotide” it is meant a naturally-occurring nucleotide, as well modified versions thereof. The term “DNA” includes double-stranded DNA, single-stranded DNA, isolated DNA such as cDNA, as well as modified DNA that differs from naturally-occurring DNA by the addition, deletion, substitution and/or alteration of one or more nucleotides as described herein.

The term “gene,” as used herein, refers to a segment of nucleic acid that encodes an individual protein or RNA and can include both exons and introns together with associated regulatory regions such as promoters, operators, terminators, 5′ untranslated regions, 3′ untranslated regions, and the like.

The term “insertion” (or “insertion mutation”), as used herein, refers to the addition of one or more nucleotides into a nucleic acid sequence (e.g., into a wild type or normal nucleic acid sequence). Insertions mutations can differ in the number of nucleotides inserted, or the nature or identity of nucleotides inserted.

A “mutation” is meant to encompass at least a nucleotide variation in a nucleotide sequence relative to a wild type or normal sequence. A mutation may include a substitution, a deletion, an inversion or an insertion. With respect to an encoded polypeptide, a mutation may be “silent” and result in no change in the encoded polypeptide sequence, or a mutation may result in a change in the encoded polypeptide sequence. For example, a mutation may result in a substitution in the encoded polypeptide sequence. A mutation may result in a frameshift with respect to the encoded polypeptide sequence.

The term “naturally-occurring,” as used herein, as applied to an object, refers to the fact that the object can be found in nature. For example, a nucleotide or nucleotide sequence that is present in an organism (including viruses) that can be isolated from a source in nature and which has not been intentionally modified by man in the laboratory is said to be naturally-occurring.

As used herein, the term “nucleic acid” has its general meaning in the art and refers to refers to a coding or non coding nucleic sequence. Nucleic acids include DNA (deoxyribonucleic acid) and RNA (ribonucleic acid) nucleic acids. Examples of nucleic acid thus include but are not limited to DNA, mRNA, tRNA, rRNA, tmRNA, miRNA, piRNA, snoRNA, and snRNA. Nucleic acids thus encompass coding and non coding region of a genome (i.e. nuclear or mitochondrial).

The term “percent (%) sequence identity,” as used herein with respect to a reference sequence is defined as the percentage of nucleotides in a candidate sequence that are identical with the nucleotides in the reference polynucleotide sequence over the window of comparison after optimal alignment of the sequences and introducing gaps, if necessary, to achieve the maximum percent sequence identity.

As used herein, the term “primer” refers to an oligonucleotide, whether occurring naturally as in a purified restriction digest or produced synthetically, which is capable of acting as a point of initiation of nucleic acid sequence synthesis when placed under conditions in which synthesis of a primer extension product which is complementary to a nucleic acid strand is induced, i.e. in the presence of different nucleotide triphosphates and a polymerase in an appropriate buffer (“buffer” includes pH, ionic strength, cofactors etc.) and at a suitable temperature. Typically, a primer has a length of 10; 11; 12; 13; 14; 15; 16; 17; 18; 19; 20; 21; 22; 23; 24; 25; 26; 27; 28; 29; or 30 nucleotides. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 57 end of the primer, with the remainder of the primer sequence being substantially complementary to the strand. The term “primer” as used herein includes all forms of primers that may be synthesized including peptide nucleic acid primers, locked nucleic acid primers, phosphorothioate modified primers, labeled primers, and the like. Primers are typically at least about 10, 15, 20, 25, 30, 35, 40, 45, 50, or more nucleotides in length. An optimal length for a particular primer application may be readily determined in the manner described in H. Erlich, PCR Technology, Principles and Application for DNA Amplification (1989). Primers can be labeled with a detectable molecule or substance, such as a fluorescent molecule, a radioactive molecule or any others labels known in the art. Labels are known in the art that generally provide (either directly or indirectly) a signal. The term “labeled” is intended to encompass direct labeling of the probe and primers by coupling (i.e., physically linking) a detectable substance as well as indirect labeling by reactivity with another reagent that is directly labeled. Examples of detectable substances include but are not limited to radioactive agents or a fluorophore (e.g. fluorescein isothiocyanate (FITC), phycoerythrin (PE), cyanine (Cy3), VIC fluorescent dye, FAM (6-carboxyfluorescein) or Indocyanine (Cy5)).

A “probe” refers to a nucleic acid that interacts with a target nucleic acid via hybridization. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases and modified sugar moieties. A probe may be fully complementary to a target nucleic acid sequence or partially complementary. A probe may be used to detect the presence or absence of a target nucleic acid. A probe or probes can be used, for example to detect the presence or absence of a mutation in a nucleic acid sequence by virtue of the sequence characteristics of the target. Probes can be labeled or unlabeled, or modified in any of a number of ways well known in the art.

The term “reverse transcription” refers to process of making a double stranded DNA molecule from a single stranded RNA template through the enzyme, reverse transcriptase.

The following terms are used herein to describe the sequence relationships between two or more polynucleotide molecules: “reference sequence,” “window of comparison,” “sequence identity,” “percent (%) sequence identity,” and “substantial identity.” A “reference sequence” is a defined sequence used as a basis for a sequence comparison; a reference sequence may be a subset of a larger sequence, for example, as a segment of a full-length cDNA or gene, or may comprise a complete cDNA or gene sequence. Generally, a reference polynucleotide sequence is at least 20 nucleotides in length, and often at least 50 nucleotides in length.

The term “selectively hybridize” or “specifically hybridize”, as used herein, refers to the ability of a particular nucleic acid sequence to bind specifically to a target nucleic acid sequence. Selective hybridization generally takes place under hybridization and wash conditions that minimize appreciable amounts of detectable binding to non-specific nucleic acids. High stringency conditions can be used to achieve selective hybridization and are known in the art and discussed herein. Typically, hybridization and washing conditions are performed at high stringency according to conventional hybridization procedures with washing conditions utilizing a solution comprising 1-3×SSC, 0.1-1% SDS at 50-70° C., optionally with a change of wash solution after about 5-30 minutes. For instance, in the present disclosure, a primer is considered to selectively hybridize to a target sequence if the primer specifically anneals to the target sequence under the digital PCR reaction conditions, e.g., in a reaction mixture comprising dNTPs, DNA polymerase and a PCR buffer comprising Mg²⁺ at a temperature typically in the range of 55-60° C. Primers having significant sequence identity to the complement of a target sequence is expected to selectively hybridize or anneal to the target sequence. Primers with at least 80% sequence identity, and at least 90%, 95%, 98% or 99% sequence identity as compared to a reference sequence over a window of comparison are considered to have significant or substantial sequence identity with the reference sequence.

The term “sequence identity” means that two polynucleotide sequences are identical (i.e. on a nucleotide-by-nucleotide basis) over the window of comparison.

The term “subject” or “patient,” as used herein, refers to a mammal who has or is suspecting of having a disease or condition. In one embodiment, the subject is suffering from a cancer. In a specific embodiment, the patient is suffering from a blood cancer.

The terms “substantial identity” or “substantially identical,” as used herein, denote a characteristic of a polynucleotide sequence, wherein the polynucleotide comprises a sequence that has at least 50% sequence identity as compared to a reference sequence over the window of comparison allowing for gaps or mismatches of several bases, which may result from genetic mutation, polymorphism, evolutionary divergence or other phenomena. Polynucleotide sequences with at least 60% sequence identity, at least 70% sequence identity, at least 80% sequence identity, and at least 90%, 95%, 98% or 99% sequence identity as compared to a reference sequence over the window of comparison are also considered to have substantial identity with the reference sequence.

As defined herein, the term “transcription” refers to the process by which an RNA molecule is produced from a nucleic acid template. A nucleic acid template may be RNA or DNA.

As used herein, the term “transcript” refers to a product of transcription.

The term “wild-type” refers to a gene or a gene product that has the characteristics of that gene or gene product when isolated from a naturally occurring source. A wild-type gene is that which is most frequently observed in a population and is thus arbitrarily designated as the “normal” or “wild-type” form of the gene. “Wild-type” may also refer to the sequence at a specific nucleotide position or positions, or the sequence at a particular codon position or positions, or the sequence at a particular amino acid position or positions. As used herein, “mutant” “modified” or “polymorphic” refers to a gene or gene product which displays modifications in sequence and or functional properties (i.e., altered characteristics) when compared to the wild-type gene or gene product. The term “mutant” “modified” or “polymorphic” also refers to the sequence at a specific nucleotide position or positions, or the sequence at a particular codon position or positions, or the sequence at a particular amino acid position or positions.

A “window of comparison”, as used herein, refers to a conceptual segment of the reference sequence of at least 15 contiguous nucleotide positions over which a candidate sequence may be compared to the reference sequence and wherein the portion of the candidate sequence in the window of comparison may comprise additions or deletions (i.e. gaps) of 20 percent or less as compared to the reference sequence (which does not comprise additions or deletions) for optimal alignment of the two sequences. The present invention contemplates various lengths for the window of comparison, up to and including the full length of either the reference or candidate sequence. Optimal alignment of sequences for aligning a comparison window may be conducted using the local homology algorithm of Smith and Waterman (Adv. Appl. Math. (1981) 2:482), the homology alignment algorithm of Needleman and Wunsch (J. Mol. Biol. (1970) 48:443), the search for similarity method of Pearson and Lipman (Proc. Natl. Acad. Sci. (U.S.A.) (1988) 85:2444), using computerized implementations of these algorithms (such as GAP, BESTFIT, FASTA, and TFASTA in the Wisconsin Genetics Software Package Release 7.0, Genetics Computer Group, 573 Science Dr., Madison, Wis.), using publicly available computer software such as ALIGN or Megalign (DNASTAR), or by inspection. The best alignment (i.e. resulting in the highest percentage of identity over the comparison window) is then selected.

### General Description

In work leading up to the present invention, a novel single multiplex digital PCR (dPCR) assay has been developed by the present inventor that utilizes insertion-specific primers that selectively detect mutated, but not wild-type, forms of a gene (such as NPM1). Such an assay enables detection of various types of insertion mutations in a single multiplex assay, as well as sensitive quantification of the types of mutations, all without the need for numerous type-specific (i.e., mutation-specific) assays.

More specifically, it has been demonstrated that a dPCR-based assay disclosed herein can detect the vast majority of NPM1 mutations in AML with a sensitivity of up to 1:10,000-1:50,000 cells. In addition to improving accessibility and easing the implementation of mutant NPM1 MRD monitoring, the present assay represents a novel application of dPCR since the test cannot be practically implemented using RQ-PCR and the approach can be applied to detecting other insertion polymorphisms in other genes.

### Methods of Detection

In one aspect, the present disclosure provides a method of detecting mutant nucleic acid (RNA, DNA or cDNA) forms of a gene in a sample, particularly where the mutations are insertion mutations at a specific site in the gene, using multiple mutation-specific primers in a single digital PCR-based assay.

The digital PCR-based assay utilizes a first primer set, a second primer, and a probe. The first primer set includes multiple insertion-specific primers that are degenerate in at least one (i.e., one or more) position corresponding to a position of an inserted nucleotide in the mutant nucleic acid molecules to be detected. By “corresponding to” it is meant that the degenerate nucleotides are positioned to form base pairs with inserted nucleotides in the mutant nucleic acids to be detected. The multiple primers in the first primer set can each pair with the second primer, thereby providing multiplex reaction pools that can generate insertion-specific amplicons in a single digital polymerase chain reaction.

### Insertion-Mutations

In some embodiments, the methods disclosed herein are directed to detecting insertion mutations that occur at a specific site in a gene, for example, insertion mutations in NPM1.

Heterozygous mutations of the NPM1 gene have been identified in approximately 35% of adult patients as well as 6.5% of children with acute myeloid leukemia (AML) (Falini et al. N. Engl. J. Med. 2005; 352: 254-266; Cazzaniga et al. Blood. 2005; 106:1419-1422). Many molecular variants of NPM1 mutations have been described to date in AML patients, with the majority falling in exon 12 (Falini et al. Blood. 2007; 109: 874-85). Many of the NPM1 mutations that have been identified in AML are characterized by simple 1- or 2-tetranucleotide insertions, a 4-base pair (bp) or 5-bp deletion combined with a 9-bp insertion, or a 9-bp deletion combined with a 14-bp insertion (Falini et al. Blood. 2007; 109: 874-85; Chen et al., Arch. Pathol. Lab Med. 2006; 130: 1687-1692). Mutations in exon 12 of the NPM1 gene often lead to frame shifts, generating an elongated protein which is retained in the cytoplasm.

NPM1 mutations are associated with high levels of bone marrow blasts, a high white blood cell (WBC) and platelet count, and fms-related tyrosine kinase 3 internal tandem duplication (FLT3-ITD) (Thiede et al. Blood. 2006; 107: 4011-4020). Patients exhibiting NPM1 mutations without FLT3 mutations showed significantly better overall and disease-free survival in this study (Thiede et al., Blood. 2006; 107: 4011-4020). NPM1 mutations are common in AML with a normal karyotype (Schnittger et al., Blood., 2005; 106: 3733-3739). Within the group of patients with AML who have a normal karyotype, various studies have shown that patients with NPM1-mutated AML had a complete remission rate similar to or significantly higher than that of patients with wild-type NPM1 AML (Boissel et al. Blood. 2005; 106: 3618-3620; Falini et al. N. Engl. J. Med. 2005; 352: 254 266; Suzuki et al. Blood. 2005; 106: 2854-2861; Dohner et al., Blood. 2005; 106: 3740-6).

In some embodiments, the methods disclosed herein are directed to detecting insertion mutations that occur at a specific site in NPM1, including insertions of one or more nucleotides between positions 863 and 864 (in exon 12), positions 860 and 861, positions 861 and 862, positions 864 and 865, positions 865 and 866, positions 866 and 867, and positions 867 and 868, as numbered based on the NPM1 coding sequence represented by SEQ ID NO: 15. As further disclosed hereinbelow, the primers can be designed to permit detection of insertions of 1, 2, 3, 4, 5 or more nucleotides.

In some embodiments, the method disclosed herein is directed to detecting insertion mutations that occur at a specific site in a gene other than NPM1, for example, EGFR.

### NPM1

The term, “NPM1”, refers to the gene encoding the nucleolar phosphoprotein B23 protein, also called numatrin or nucleophosmin 1, referenced as OMIM 164040 and NCBI Gene ID: 4869. An Ensembl Transcript ID of the wild-type NPM1 mRNA transcript is ENST00000517671.5. The coding sequence of the NPM1 gene is set forth in SEQ ID NO: 15. The exon numbering referenced herein is based on ENST00000517671.5, and the nucleotide position numbering is based on SEQ ID NO: 15.

The NPM1 gene encodes a phosphoprotein which moves between the nucleus and the cytoplasm. The gene product is thought to be involved in several processes including regulation of the ARF/p53 pathway.

The NPM1 gene is located on chromosome 5q35. Disruption of NPM1 by reciprocal chromosomal translocation is involved in several hematolymphoid malignancies (Falini et al., Hematologica. 2007; 92(4): 519-532). These translocations result in the formation of various fusion proteins that retain the N-terminus of nucleophosmin and have been associated with neoplastic conditions including NPM-anaplastic large cell lymphoma kinase (NPM-ALK) in anaplastic large cell lymphoma (Morris et al., Science, 1994; 263:1281-1284), NPM-retinoic acid receptor-alpha (NPM-RARu) in acute promyelocytic leukemia (Redner et al., Blood 1996; 87: 882-88), and NPM-myelodysplasia/myeloid leukemia factor 1 (NPM-MLF1) in AML/myclodysplastic syndrome (Yoneda-Kato et al., Oncogene 1996; 12: 265-275).

To date, the NPM1 gene is the most clinically relevant marker for molecular monitoring in cytogenetically normal karyotype Acute Myeloid Leukemia (CN-AML) because it is present in 53% of CN-AML patients. NPM1 mutations (NPM1mut) are typically 4-nucleotide frameshift insertions in exon 12. The patient-to-patient consistency and relative stability through diagnosis and successive relapse in each patient have rendered NPM1mut an ideal and useful molecular MRD marker.

### First Primer Set

The “first primer set” used in a digital PCR assay disclosed herein is a set or collection of multiple insertion-specific primers.

By “insertion-specific” it is meant that a primer binds, i.e., hybridizes or anneals, to a mutant nucleic acid of a gene containing an insertion mutation, but not to an otherwise identical nucleic acid without the insertion (e.g., a wild type gene). Therefore, the multiple insertion-specific primers in a first primer set are also considered herein as specifically annealing or specifically hybridizing to mutant nucleic acids containing insertion mutations.

In some embodiments, the multiple insertion-specific primers in a first primer set comprise nucleotides at two to four adjacent positions corresponding to positions of inserted nucleotides in mutant nucleic acids of a target gene, with at least one of the two to four positions being degenerate, but are otherwise identical with a target gene sequence without an insertion mutation. In order to provide a set of primers that can amplify as many insertion mutations as possible, multiple (i.e., 2, 3, 4, 5 or more) positions can be made degenerate in the primer design, e.g., primers containing NN, NNN, NNNN, NNNN, NNNNN, or the like, wherein N at each position can independently be A, C, G, T, dI, 5NI, or other nucleotides and modifications thereof, and wherein one or more of the positions or all of the positions are made degenerate. The term “independently” as used herein means that adjacent nucleotides in a sequence formula of NN, NNN or NNNN are not necessarily the same or different.

In some embodiments, a first primer set includes primers that contain degenerate nucleotides at 4 consecutive positions, i.e., NNNN, wherein N at each position represents a degenerate oligonucleotide and can independently be A, C, G or T, which provides a primer set consisting of 4⁴ primers, differing from each other at a position denoted by N.

In some embodiments, a first primer set includes primers that contain degenerate nucleotides at 3 consecutive positions, i.e., NNN, wherein N at each position represents a degenerate oligonucleotide and can independently be A, C, G or T, which provides a primer set consisting of 4³ primers, differing from each other at a position denoted by N.

In some embodiments, a first primer set includes primers that contain degenerate nucleotides at 2 adjacent positions, i.e., NN, wherein N at each position represents a degenerate oligonucleotide and can independently be A, C, G or T, which provides a primer set consisting of 4² primers, differing from each other at a position denoted by N.

In some embodiments, a first primer set includes a combination of primers that contain degenerate nucleotides at 4, 3 and 2 consecutive positions, respectively, i.e., a combination of primers having NNNN, NNN, or NN, wherein N at each position represents a degenerate oligonucleotide and can independently be A, C, G or T.

In some embodiments, the primers in a first primer set can be designed to comprise nucleotides at two to four adjacent positions corresponding to positions of inserted nucleotides in mutant nucleic acids of a target gene, with at least one of the two to four positions being degenerate, but one or more positions being not degenerate and fixed to a nucleotide selected from A, C, G or T.

In some embodiments, the primers in a first primer set can be designed based on partial degeneracy. For example, instead of having A, C, G or T as four possible nucleotides for one degenerate position in primers, the primers can be designed and synthesized to have Y (i.e., C or T), W (i.e., A or T), or R (A or G) at one position.

When referring to a first primer set as having a degenerate nucleotide at one or more positions, it is meant that the multiple primers in the first primer set are degenerate at such one or more positions and differ from each other in such one or more positions but are otherwise identical. Such primer set can be made synthetically by providing a mixture of nucleotides of choice for a selected position during synthesis. In some embodiments, the mixture of nucleotides is a mixture of A, C, G, T, dI, 5NI and other modified nucleotides, a mixture of A, C, G and T, a mixture of C and T, a mixture of A and T, or a mixture of A and G. In some embodiments, the selected nucleotides in a mixture are at a predetermined ratio relative to each other. In some embodiments, the amounts of the selected nucleotides in the mixture are equal relative to each other; and in other embodiments, the amounts of the selected nucleotides in the mixture are not equal relative to each other, and are predetermined to favor detection of common insertion mutations.

In various embodiments, the primers in a first primer set are designed to have the one or more degenerate oligonucleotides flanked by sequences that complement or substantially complement (or in other words, sequences that are identical or substantially identical to the complement of) a target wild type gene sequence. The lengths of the sequences flanking the degenerate oligonucleotide(s) may vary and can be determined by those skilled in the art. Generally speaking, the primers in a first primer set can be of a length of at least 14, 15, 16, 17, 18, 19, 20, 21, 22, 23, 24, 25, 26, 27 or more nucleotides, and there can be at least 3, 4, 5, 6, 7, 8, 9, 10, 11, 12, 13, 14, or more nucleotides on the 5′ side of the degenerate oligonucleotide(s), and at least 3, 4, 5, 6, 7, 8, 9, 10, 11, 12, 13, 14 or more nucleotides on the 3′ side of the degenerate oligonucleotide(s).

In one embodiment, the first primer set is a set of multiple primers which are specific for insertion mutations of the NPM1 gene in exon 12 at nucleotide position 863 (i.e., insertion between positions 863 and 864), with the nucleotide numbering based on the NPM1 sequence set forth in SEQ ID NO: 15. In other embodiments, the first primer set is a set of multiple primers which are specific for insertion mutations of the NPM1 gene at another site (e.g., between positions 859 and 860, positions 860 and 861, positions 861 and 862, positions 862 and 863, positions 864 and 865, positions 865 and 866, positions 866 and 867, and positions 867 and 868).

In one embodiment, the primers in the first primer set are designed to be specific for four nucleotide insertion mutations in the NPM1 gene. Reported four nucleotide insertion mutations in the NPM1 gene include “YWTG” (c.863_864insYWTG), wherein Y refers to C or T, and W refers to A or T; “TCTG” (c.863_864insTCTG), referred to as a type A mutation; “CATG” (c.863_864insCATG), referred to as a type B mutation; and “CCTG” (c.863_864insCCTG), also referred to as a type D mutation. Design of primers that include four degenerate nucleotides (i.e., NNNN) permits detection of all these reported four nucleotide insertion mutations in the NPM1 gene in a single digital PCR assay.

In one embodiment, the first primer set includes primers with 4 adjacent nucleotides (NNNN) specific for insertion mutations at position 863 in the NPM1 gene, wherein N at each position can be independently A, T, C, G, dI or 5NI, and wherein at least 2 of the 4 nucleotides are degenerate.

In some embodiments, the primers containing 4 adjacent nucleotides (“NNNN”) specific for insertion mutations at position 863 in the NPM1 gene include a sequence of at least 4-14 nucleotides (i.e., at least 4, 5, 6, 7, 8, 9, 10, 11, 12, 13, or 14 nucleotides) on the 5′ side of NNNN, which sequence fully or substantially complements the sequence on the 5′ side of position 863 (inclusive) in the NPM1 gene; and includes a sequence of at least 4-14 nucleotides (i.e., at least 4, 5, 6, 7, 8, 9, 10, 11, 12, 13, or 14 nucleotides) on the 3′ side of NNNN, which sequence fully or substantially complements the sequence on the 3′ side of position 864 (inclusive) in the NPM1 gene.

In a specific embodiment, the first primer set is a reverse primer set with a sequence that is substantially identical to (5′-CTTCCTCCACTGCNNNNCAGA-3′) (SEQ ID NO: 3), wherein N at each position is independently A, T, C, G, dI or 5NI, with 2, 3 or 4 of the N's being degenerate. In a specific embodiment, N at each position is independently A, T, C or G. The term “independently” as used herein means that a sequence of “NNNN” does not necessarily refer to four identical nucleotides in a row.

In yet another embodiment, the multiple primers in the first primer set are reverse primers with a sequence that is substantially identical to (5′-CTTCCTCCACTGCCAWRCAGA-3′) (SEQ ID NO: 9), wherein R is A or G, and W is A or T.

In some embodiments, the first primer set is a forward primer set with a sequence that is substantially identical to (5′-TCTGNNNNGCAGTGGAGGAAG-3′) (SEQ ID NO: 12), wherein N at each position is independently A, T, C, G, dI or 5NI. In a specific embodiment, N at each position is independently A, T, C or G. In a specific embodiment, the first primer set is prepared such that N at each position is independently 30% A, 40% T, 10% C, 20% G.

In some embodiments, at least one or two out of the four positions denoted as “N” in the first primer set are fixed to a nucleotide selected from the group consisting of A, T, G or C, i.e., not degenerate at such one or two positions. Such design permits detection of common 4-nucleotide insertions that share one or two inserted nucleotides.

In some embodiments, the multiple primers in the first primer set are forward primers with a sequence that is substantially identical to (5′-TCTGYWTGGCAGTGGAGGAAG-3′) (SEQ ID NO: 13), wherein Y is C or T and W is A or T. In some embodiments, Y is 90% C and 10% T, Y is 80% C and 20% T, Y is 70% C and 30% T or Y is 60% C and 40% T.

In some embodiments, the multiple primers in the first primer set are reverse primers represented by the sequence 5′-CTTCCTCCACTGCCAWRCAGA-3′ (SEQ ID NO: 14), wherein W is A or T and R is A or G.

### Second Primer

The assay disclosed herein utilizes a second primer in addition to a first primer set. The second primer is a primer that produces amplicons of about 60-200 base pairs (bp) when used with the first primer set in a Polymerase Chain Reaction (PCR). In a specific embodiment the second primer is a primer that produces an amplicon of about 75-150 base pairs (bp) when used with the first primer set in a PCR. In a specific embodiment the second primer is a primer that produces an amplicon of about 85-120 base pairs (bp) when used with the first primer set in a PCR. In a specific embodiment the PCR is reverse transcriptase PCR (RT-PCR), reverse transcriptase quantitative PCR (RQ-PCR or real-time quantitative PCR), digital PCR or any other PCR methods known in the art.

In some embodiments, the second primer and the first primer set produce amplicons of about 75 bp, 80 bp, 85 bp, 90 bp, 95 bp, 100 bp, 105 bp, 110 bp, 115 bp, 120 bp, 125 bp, 130 bp, 140 bp, 145 bp, 150 bp, 155 bp, 160 bp, 165 bp, 170 bp, 175 bp, 180 bp, 185 bp, 190 bp, 195 bp or 200 bp.

Generally speaking, a second primer can be of a length of at least 14, 15, 16, 17, 18, 19, 20, 21, 22, 23, 24, 25, 26, 27 or more nucleotides,

For detecting insertion mutations in exon 12 of the NPM1 gene, in embodiments where the second primer is a forward primer, the second primer can include a nucleotide sequence that is specific for (i.e., specifically hybridizes or anneals to, or complements fully or substantially) a sequence in exon 9, 10, 11 or a combination of sequences in exons 9, 10 and/or 11 of NPM1. The phrase “a combination of sequences in exons 9, 10 and/or 11” as used herein means that a second primer can hybridize to sequences from more than one exon. For instance, in one embodiment, a primer anneals to a nucleotide sequence that spans the exon-exon boundary of exon 9 and 10; and in another embodiment, a primer anneals to a nucleotide sequence that spans the exon-exon boundary of exon 10 and 11. In embodiments where the second primer is a reverse primer, the second primer can include a nucleotide sequence that is specific for (i.e., specifically hybridizes or anneals to, or complements fully or substantially) a sequence in exon 12 of NPM1. Primers can also be designed to anneal to an intron sequence as appropriate.

In a specific embodiment, the second primer comprises a sequence substantially identical to (5′-GAAGAATTGCTTCCGGATGACT-3′) (SEQ ID NO: 1).

### Probe

The probe used in assays disclosed herein is an oligonucleotide that hybridizes to the target nucleic acid. In some embodiments, the probe hybridizes to a sequence in the target nucleic acid located between the sequence to which the first primer set hybridizes (e.g., a sequence encompassing the site of insertions) and the sequence to which the second primer hybridizes. In some embodiments, the probe is between 15-25 bases long. In a specific embodiment, a probe suitable for use in assays for detecting insertion mutations in NPM1 comprises the sequence, 5′-ACCAAGAGGCTATTCAA-3′ (SEQ ID NO: 2).

In some embodiments, the probe comprises modifications, such as one or more chromophores, or a 3′-terminus modification that makes the probe non-extendable by nucleic acid polymerases. In some embodiments, the probe comprises a detectable label.

In a specific embodiment, the probe comprises a fluorophore and a quencher. Prior to an amplification reaction, a probe has low fluorescence due to the presence of a quencher molecule. During the amplification step of PCR, the probe that is hybridized to the template (target nucleic acid) is hydrolyzed resulting in the release of the quencher and an increase in the fluorescence signal.

As used herein, a “fluorophore” (or “fluorescing species”) refers to any species possessing a fluorescent property when appropriately stimulated. The stimulation that elicits fluorescence is typically illumination; however, other types of stimulation (e.g., collisional) are also considered herein. Examples of fluorophores and quenchers are well-known in the art (e.g. as described in U.S. Pat. No. 8,945,515, contents of which are incorporated in its entirety).

In some exemplary embodiments, the fluorescent label is selected from the group consisting of fluorescein isothiocyanate (FITC), phycoerythrin (PE), cyanine (Cy3), Indocyanine (Cy5), VIC fluorescent dye and FAM (6-carboxyfluorescein), and wherein the quencher is Minor Groove Binder (MGB).

In some embodiments, the probe comprises a FAM label and a Minor Groove Binder (MGB) quencher.

### Sample

The term “sample” or “patient sample” as used herein includes biological samples such as tissues and bodily fluids. “Bodily fluids” may include, but are not limited to, blood, serum, plasma, saliva, cerebral spinal fluid, pleural fluid, tears, lactal duct fluid, lymph, sputum, urine, amniotic fluid, and semen. A sample may include a bodily fluid that is “acellular.” An “acellular bodily fluid” includes less than about 1% (w/w) whole cellular material. Plasma or serum are examples of acellular bodily fluids. A sample may include a specimen of natural or synthetic origin (i.e., a cellular sample made to be acellular).

In some embodiments, samples suitable for use in this method comprise biological samples comprising RNA transcripts. A sample may be, for example, a blood sample (e.g., a sample obtained from a fingerstick, or from venipuncture, or an arterial blood sample), a urine sample, a biopsy sample, a tissue slice, stool sample, or other biological sample. In a specific embodiment, a blood sample comprises whole blood.

In some embodiments, samples can be biopsy samples from a subject. Mononuclear cells may be isolated from said biopsy. Said mononuclear cells may be washed and RNA may be isolated from said cells using methods known in the art, including but not limited to, using the RNeasy extraction kit (Qiagen) per the manufacturer's specifications.

In other embodiments, the sample is a sample where RNA transcripts in an original sample have been reverse-transcribed into cDNA molecules.

In a specific embodiment, the sample is from a patient suspected of having a cancer. In a specific embodiment, the cancer is a blood cancer which can include, for example, non-Hodgkin's lymphoma, acute promyelocytic leukemia, myelodysplastic syndrome, acute lymphocytic leukemia (ALL) and acute myelogenous leukemia (AML).

In another embodiment, the sample is from a cancer patient who has already undergone an anti-cancer therapy. In a specific embodiment, the present method detects the presence of minimal residual disease (MRD) in a patient treated with an anti-cancer therapy.

### Digital PCR

Digital PCR (dPCR) is used in this disclosure to detect and/or genetic variants. dPCR is described in the art, for example, in Baker M., Nature Methods 9, 541-544 (2012). In a digital PCR, a sample mixed with PCR primers is partitioned into hundreds to millions of droplets or wells (depending on the technological platform) each containing a single copy or a few copies of a target template. The capture or isolation of individual nucleic acid molecules is achieved in micro well plates, capillaries, the dispersed phase of an emulsion, and arrays of miniaturized chambers, as well as on nucleic acid binding surfaces. PCR is performed in the separated regions in parallel. The partitioning of the sample allows one to estimate the number of different molecules by assuming that the molecule population follows the Poisson distribution. As a result, each region will contain “O” or “1” molecules, or a negative or positive PCR reaction, respectively. After PCR amplification, nucleic acids may be quantified by counting the regions that contain PCR end-product, which are the result of positive reactions. Reliance of dPCR upon reaction end-points rather than reaction kinetics confers an ability to remain quantitative even under highly multiplexed assay conditions.

For the embodiments of this disclosure, any method that partitions an entire PCR reaction into smaller partitions, followed by submitting these individual partitions to enzymatic and cycling conditions well known in the art to perform PCR, can be used. Partitions can be comprise oil droplet partitions, physical partitions on a chip, or a multi-well plate..

Typical PCR enzymatic and cycling conditions can be employed. For example, an initial polymerase activation at 95° C. (e.g., for 10 minutes) is followed by 30-45 cycles of denaturation (95° C.), and annealing and extension (at a temperature in the range of 58-63° C.). Reactions are terminated with a 98° C. incubation and are held at 10° C. until analysis.

In some embodiments droplet digital PCR may be performed using the RainDrop platform (RainDance Technologies), QX-200 platform (BioRad), QuantStudio 3D (ThermoFisher) or the Biomark System (Fluidigm). Results are analyzed according to the methods known in the art.

In some embodiments, droplet digital PCR is performed using either the RainDrop platform. For the RainDrop platform, reaction mixtures consist of 1× Taqman Genotyping Master Mix (Life Technologies), 1× Droplet Stabilizer (RainDance Technologies), 500 nM of each forward and reverse primer for NPM1 and ABL1 and 250 nM of each probe. After generation of droplet partitions, 10 minutes of polymerase activation (95° C.) are followed by 45 cycles of denaturation (95° C.) and annealing and extension (58° C. for multiplex assay; other temperatures indicated in Table 1) using BioMetra TAdvanced Thermocycler (Gottingen, Germany). Reactions are terminated with a 98° C. incubation and are held at 10° C. until analysis.

In some embodiments, droplet digital PCR is performed using either the QX-200 platform. For the QX-200 platform, reactions are performed using the ddPCR Supermix for Probes (No dUTP) (BioRad) and processed according to manufacturer's protocols on a C1000 Touch Thermal Cycler (BioRad) using the amplification protocol and primer/probe concentrations as above. Droplet positivity is quantified using the manufacturer's software, either RainDrop Analyst II v1.1 (RainDance Technologies) or QuantaSoft v1.0 (BioRad).

### Quantifying Mutant Nucleic Acids

In some embodiments, the method further comprises quantifying mutant forms of a target gene that are detected in a digital PCR, e.g., quantifying NPM1 nucleic acids (e.g., transcripts) containing insertion mutations at position 863.

In some embodiments, the method further comprises detecting and quantifying nucleic acids (e.g., transcripts) of a second gene in a digital PCR, and generating a ratio of mutant NPM1 nucleic acids to nucleic acids of the second gene. The second gene can be selected from any gene that is stably expressed across cell types. In some embodiments, the second gene is selected from the group consisting of GAPDH, wild-type NPM1, (3-actin and ABL1.

In a specific embodiment, the second gene is ABL1. In still another embodiment, a second probe substantially identical to 5′-CATTTTTGGTTTGGGCTTC-3′ (SEQ ID NO: 8) is used to quantify ABL1, wherein the second probe has a second fluorescent label different from the fluorescent label of the first probe.

In one embodiment, detection of any mutated NPM1 transcript greater than or equal to the detection limit by dPCR is indicative of cancer or residual cancer cells. In a specific embodiment of the present disclosure, the detection limit for the dPCR is 0, 1, 2, 5, 10, 50, 100 or 500 mutated NPM1 transcripts per 10,000 ABL1 (or other normalizer) transcripts.

In an alternative embodiment, it is envisioned that residual disease ratios can be represented as a percentage of mutated NPM1 transcripts as a fraction of any normalizer gene including, but not limited to, wild type NPM1 copies, total NPM1 (mutant and wild-type) transcript copies, GUSB transcript copies, or GAPDH transcript copies or any stably expressed housekeeping gene. Further still, mutated NPM1 transcript copies may be represented per volume of blood or bone marrow or blood products (e.g. plasma) or per total number of cells.

Thus, the limit of detection can be established for any of above residual disease ratios and a sample is considered positive if it is greater than or equal to the detection limit or any other mathematical or statistical approach that represents the probability that the signal is above background values expected in healthy patients and defines thresholds thereof.

In some embodiments, the cancer is a blood cancer. In another embodiment, the blood cancer is selected from the group consisting of non-Hodgkin lymphoma, acute promyelocytic leukemia, myelodysplastic syndrome, acute lymphocytic leukemia and acute myelogenous leukemia. In yet another embodiment, the sample is a sample of a cancer patient who has undergone cancer therapy.

### Kits.

In a further aspect, this disclosure provides a kit useful for detecting and/or quantifying mutant nucleic acids of the NPM1 gene in a sample. The kit includes a first primer set, a second primer, and a probe, each as described hereinabove.

In some embodiments, the kit further comprises a probe for a second gene. In a specific embodiment, said second gene is ABL1. In one embodiment, the probe for the ABL1 gene includes a nucleotide sequence that is substantially identical to (5′CATTTTTGGTTTGGGCTTC-3′) (SEQ ID NO: 8).

In an embodiment, instructions for using the kit are provided with the kit.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The present description is further illustrated by the following examples, which should not be construed as limiting in any way.

### EXAMPLES

### Example 1: Materials and Methods

Primary Sample Isolation and Cell Culture

Mononuclear cell (MNC) isolation was performed using Ficoll-Paque (Pharmacia Biotech) density gradient centrifugation. GM12878 cell line (Coriell) were cultured in RPMI medium supplemented with 15% FBS and L-Glutamine (2 mM). OCI-AML3 cells were cultured in alpha-MEM supplemented with 20% heat-inactivated FBS.

RNA Extraction

Cells were washed with Dulbecco's phosphate-buffered saline (D-PBS) and total RNA was isolated using the RNeasy extraction kit (Qiagen) as per the manufacturer's specifications.

Primers, Probes, Synthetic Targets, and Plasmid Standards.

NPM1 mutation detection was performed using primers and probes described in Gorello et al (Gorello et al. 2006) with modifications when performing digital PCR. Multiplex digital PCR reactions for NPM1 detection consisted of a common forward primer (5′-GAAGAATTGCTTCCGGATGACT-3′) (SEQ ID NO: 1) and probe (5′FAM-ACCAAGAGGCTATTCAA-MGB-3′) (SEQ ID NO: 2) as described (Gorello et al., Leukemia (2006), 20 (6): 1103-8) combined with a degenerate reverse primer (5′-CTTCCTCCACTGCNNNNCAGA-3′) (SEQ ID NO: 3) adapted from Gorello et al. (Leukemia (2006), 20 (6): 1103-8) to generate a multiplex reaction pool (where “N” is a mixture of A, C, G, and T). Additional primers and probes are indicated in Table 1. Probes were synthesized by ThermoFisher and primers by ThermoFisher or Integrated DNA Technologies. Synthetic targets for rare NPM1 subtypes were generated as gBlocks (Integrated DNA Technologies). Plasmid reference standards (Ipsogen) were used for quantification by RT-qPCR. A synthetic NPM1 mutated target pool and NPM1 wild-type (GeneArt fragments) were obtained from ThermoFisher. Sequences for all synthetic DNA targets are listed on Table 2. Plasmid reference standards were used for quantification by RT-qPCR (Qiagen).

Reverse Transcription.

cDNA was synthesized from 500 ng total RNA with SuperScript VILO cDNA Synthesis (Life Technologies). Reactions were incubated at 25° C. for 10 minutes, 42° C. for 60 minutes, and at 85° C. at 5 minutes. Samples were diluted with 30 μl of H2O to a final concentration of 10 ng/μl of RNA equivalent.

Real-Time Quantitative PCR.

Real time quantitative PCR (RT-qPCR) reactions were performed on a QuantStudio 5 platform (Applied Biosystems) as described in Gorello et al. (2006), supra. Annealing temperature varies according to target.

Digital Droplet PCR.

Droplet digital PCR was performed using either the RainDrop platform (RainDance Technologies) or QX-200 platform (BioRad). For the RainDrop platform, reaction mixtures consisted of 1× Taqman Genotyping Master Mix (Life Technologies), 1× Droplet Stabilizer (RainDance Technologies), 500 nM of each forward and reverse primer for NPM1 and ABL1 and 250 nM of each probe. After generation of droplet partitions, 10 minutes of polymerase activation (95° C.) were followed by 45 cycles of denaturation (95° C.) and annealing and extension (58° C. for multiplex assay; other temperatures per usual, using BioMetra TAdvanced Thermocycler. Reactions were terminated with a 98° C. incubation and held at 10° C. until analysis. For the QX-200 platform, reactions were performed using the ddPCR Supermix for Probes (No dUTP) (BioRad) and processed according to manufacturer's protocols and the C1000 Touch Thermal Cycler (BioRad) using the amplification protocol and primer/probe concentrations as above. Droplets were quantified using the manufacturer's software and/or FlowCore packages (BioConductor).

### Example 2: Development of Massively Multiplex Digital PCR Assay for NPM1 Mutations

Multiplex insertion-specific primers covering all known and theoretical 4 nt insertions were synthesized for the most common insertion site (position 863) (Table 1).

TABLE 1
Primers and probes specific to this study.

| | | Annealing |
|---|---|---|
| | | Temp |
| Description | Reverse Primer | (° C.) |
| NPM1 multiplex | 5′-CTTCCTCCACTGCNNNNCAGA-3′ | 58 |
| reverse primer | (SEQ ID NO: 4) | |
| NPM1-dI reverse | 5′-CTTCCTCCACTGC(dI)₄CAGA-3′ | 58 |
| primer | (SEQ ID NO: 5) | |
| NPM1-5NI reverse | 5′-CTTCCTCCACTGC(5NI)₄CAGA-3′ | 58 |
| primer | (SEQ ID NO: 6) | |
| NPM1 common | 5′-GAAGAATTGCTTCCGGATGACT-3′ | 58 |
| forward primer | (SEQ ID NO: 7) | |
| c.860_863dupTCTG | 5′-CTTCCTCCACTGCCAGACAGA-3′ | 62 |
| (Type A) | (SEQ ID NO: 16) | |
| c.863_864insCATG | 5′-CCTCCACTGCCATGCAGAG-3′ | 60 |
| (Type B) | (SEQ ID NO: 17) | |
| c.863_864insCCTG | 5′-CCTCCACTGCCAGGCAGA-3′ | 61 |
| (Type D) | (SEQ ID NO: 18) | |
| c.863_864insCTTG | 5′-CCTCCACTGCCAAGCAGAG-3′ | 62 |
| (Type DD1) | (SEQ ID NO: 19) | |
| c.863_864insTATG | 5′-CTTCCTCCACTGCCATACAGA-3′ | 60 |
| | (SEQ ID NO: 20) | |
| c.863_864insTCGG | 5′-CTCCACTGCCCGACAGAGA-3′ | 60 |
| | (SEQ ID NO: 21) | |
| c.863_864insTAAG | 5′-CTTCCTCCACTGCCTTACAGAGA-3′ | 63 |
| | (SEQ ID NO: 22) | |
| c.863_864insCGTG | 5′-CCTCCACTGCCACGCAG-3′ | 60 |
| | (SEQ ID NO: 23) | |
| c.863_864insTTTG | 5′-TCCTCCACTGCCAAACAGA-3′ | 60 |
| | (SEQ ID NO: 24) | |
| c.863_864insCAAA | 5′-TCCTCCACTGCTTTGCAGA-3′ | 60 |
| | (SEQ ID NO: 25) | |
| c.863_864insTAGG | 5′-CTTCCTCCACTGCCCTACAGAG-3′ | 60 |
| | (SEQ ID NO: 26) | |
| c.863_864insCTCG | 5′-CCTCCACTGCCGAGCAGA-3′ | 60 |
| | (SEQ ID NO: 27) | |
| c.864_864delinsCCGTT | 5′-TCCTCCACTGAACGGCAGA-3′ | 60 |
| | (SEQ ID NO: 28 | |
| c.865_866insCAGC | 5′-CTTCCTCCACTGCCTGGCAGA-3′ | 60 |
| | (SEQ ID NO: 29) | |
| c.863_864insGCCG | 5′-CTTCCTCCACTGCCCGGCAGA-3′ | 61 |
| | (SEQ ID NO: 30) | |

Sequences for reverse primers are listed for all NPM1mut subtypes tested as well as massively multiplex NPM1mut and NPM1-Universal assays (which amplifies both wild-type and mutant NPM1). Annealing Temperature (Tm) is indicated for universal, multiplex, NPM1 c.863_864insCTTG, NPM1 c.865_866insCAGC and NPM1 c.863_864insGCCG assays.

TABLE 1
Probes:

| | | Annealing |
|---|---|---|
| | | Temp |
| Probe | Reverse Primer | (° C.) |
| NPM1 probe | 5′FAM-ACCAAGAGGCTATTCAA- | 58 |
| | MGB-3′ (SEQ ID NO: 2) | |
| ABL1 probe | 5′VIC-CATTTTTGGTTTGGGCTT | 58 |
| | C-MGB-3′ (SEQ ID NO 8) | |

TABLE 2
Sequence of synthetic NPM1 mutant transcripts

| Insertion | Synthetic sequence |
|---|---|
| NPM1- | GACCTAGTTCTGTAGAAGACATTAAAGCAAAAATGCA |
| c863del864G_ | AGCAAGTATAGAAAAAGGTGGTTCTCTTCCCAAAGTG |
| insCCGTT | GAAGCCAAATTCATCAATTATGTGAAGAATTGCTTCC |
| | GGATGACTGACCAAGAGGCTATTCAAGATCTCTGCCG |
| | TTCAGTGGAGGAAGTCTCTTTAAG |
| | (SEQ ID NO 31) |
| NPM1-c863_ | GACCTAGTTCTGTAGAAGACATTAAAGCAAAAATGCA |
| 864insCAAA | AGCAAGTATAGAAAAAGGTGGTTCTCTTCCCAAAGTG |
| | GAAGCCAAATTCATCAATTATGTGAAGAATTGCTTCC |
| | GGATGACTGACCAAGAGGCTATTCAAGATCTCTGCAA |
| | AGCAGTGGAGGAAGTCTCTTTAAG |
| | (SEQ ID NO: 32) |
| NPM1-c863_ | GACCTAGTTCTGTAGAAGACATTAAAGCAAAAATGCA |
| 864insCGTG | AGCAAGTATAGAAAAAGGTGGTTCTCTTCCCAAAGTG |
| | GAAGCCAAATTCATCAATTATGTGAAGAATTGCTTCC |
| | GGATGACTGACCAAGAGGCTATTCAAGATCTCTGCGT |
| | GGCAGTGGAGGAAGTCTCTTTAAG |
| | (SEQ ID NO: 33) |
| NPM1-c863_ | GACCTAGTTCTGTAGAAGACATTAAAGCAAAAATGCA |
| 864insCTCG | AGCAAGTATAGAAAAAGGTGGTTCTCTTCCCAAAGTG |
| | GAAGCCAAATTCATCAATTATGTGAAGAATTGCTTCC |
| | GGATGACTGACCAAGAGGCTATTCAAGATCTCTGCTC |
| | GGCAGTGGAGGAAGTCTCTTTAAG |
| | (SEQ ID NO: 34) |
| NPM1-c863_ | GACCTAGTTCTGTAGAAGACATTAAAGCAAAAATGCA |
| 864insTAGG | AGCAAGTATAGAAAAAGGTGGTTCTCTTCCCAAAGTG |
| | GAAGCCAAATTCATCAATTATGTGAAGAATTGCTTCC |
| | GGATGACTGACCAAGAGGCTATTCAAGATCTCTGTAG |
| | GGCAGTGGAGGAAGTCTCTTTAAG |
| | (SEQ ID NO: 35) |
| NPM1-c863_ | GACCTAGTTCTGTAGAAGACATTAAAGCAAAAATGCA |
| 864insTATG | AGCAAGTATAGAAAAAGGTGGTTCTCTTCCCAAAGTG |
| | GAAGCCAAATTCATCAATTATGTGAAGAATTGCTTCC |
| | GGATGACTGACCAAGAGGCTATTCAAGATCTCTGTAT |
| | GGCAGTGGAGGAAGTCTCTTTAAG |
| | (SEQ ID NO: 36) |
| NPM1-c863_ | GACCTAGTTCTGTAGAAGACATTAAAGCAAAAATGCA |
| 864insTCGG | AGCAAGTATAGAAAAAGGTGGTTCTCTTCCCAAAGTG |
| | GAAGCCAAATTCATCAATTATGTGAAGAATTGCTTCC |
| | GGATGACTGACCAAGAGGCTATTCAAGATCTCTGTCG |
| | GGCAGTGGAGGAAGTCTCTTTAAG |
| | (SEQ ID NO: 37) |
| NPM1-c863_ | GACCTAGTTCTGTAGAAGACATTAAAGCAAAAATGCA |
| 864insTTTG | AGCAAGTATAGAAAAAGGTGGTTCTCTTCCCAAAGTG |
| | GAAGCCAAATTCATCAATTATGTGAAGAATTGCTTCC |
| | GGATGACTGACCAAGAGGCTATTCAAGATCTCTGTTT |
| | GGCAGTGGAGGAAGTCTCTTTAAG |
| | (SEQ ID NO: 9) |
| NPM1- | GACCTAGTTCTGTAGAAGACATTAAAGCAAAAATGCA |
| c863ins4N | AGCAAGTATAGAAAAAGGTGGTTCTCTTCCCAAAGTG |
| | GAAGCCAAATTCATCAATTATGTGAAGAATTGCTTCC |
| | GGATGACTGACCAAGAGGCTATTCAAGATCTCTGNNN |
| | NGCAGTGGAGGAAGTCTCTTTAAGAAAATAGTTTAAA |
| | CAATTTGTTAAAAAATTTTCCGTCTTATTTCATTTCT |
| | GTAACAGTTGATATCTGGCTGTCCTTTTTATAATGCA |
| | GAGTGAGAACTTTCCCTACCGTGTTTGATAAATGTTG |
| | TCCAGGTTCTATTGCCAAGAATGTGTTGT |
| | (SEQ ID NO: 11) |
| NPM1-wt | GACCTAGTTCTGTAGAAGACATTAAAGCAAAAATGCA |
| | AGCAAGTATAGAAAAAGGTGGTTCTCTTCCCAAAGTG |
| | GAAGCCAAATTCATCAATTATGTGAAGAATTGCTTCC |
| | GGATGACTGACCAAGAGGCTATTCAAGATCTCTGGCA |
| | GTGGAGGAAGTCTCTTTAAGAAAATAGTTTAAACAAT |
| | TTGTTAAAAAATTTTCCGTCTTATTTCATTTCTGTAA |
| | CAGTTGATATCTGGCTGTCCTTTTTATAATGCAGAGT |
| | GAGAACTTTCCCTACCGTGTTTGATAAATGTTGTCCA |
| | GGTTCTATTGCCAAGAATGTGTTGT |
| | (SEQ ID NO: 10) |

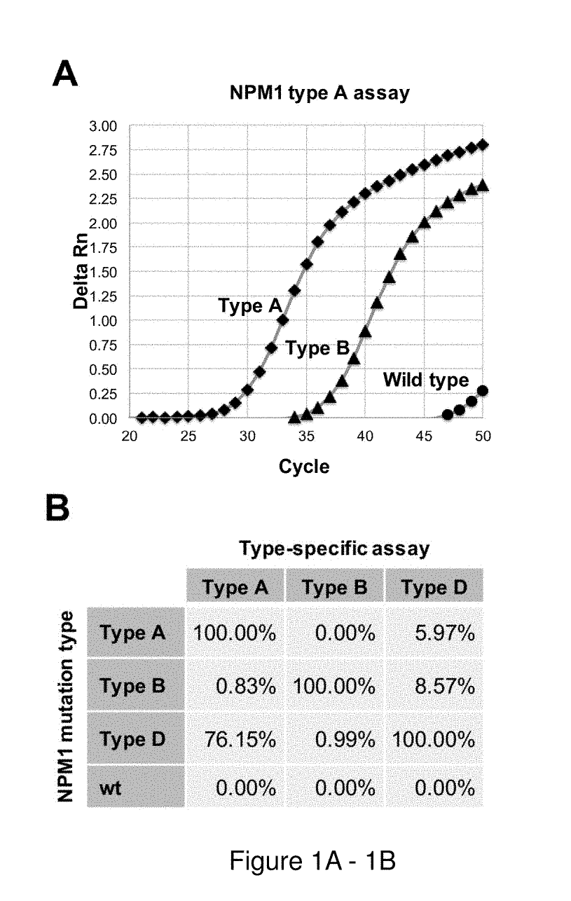

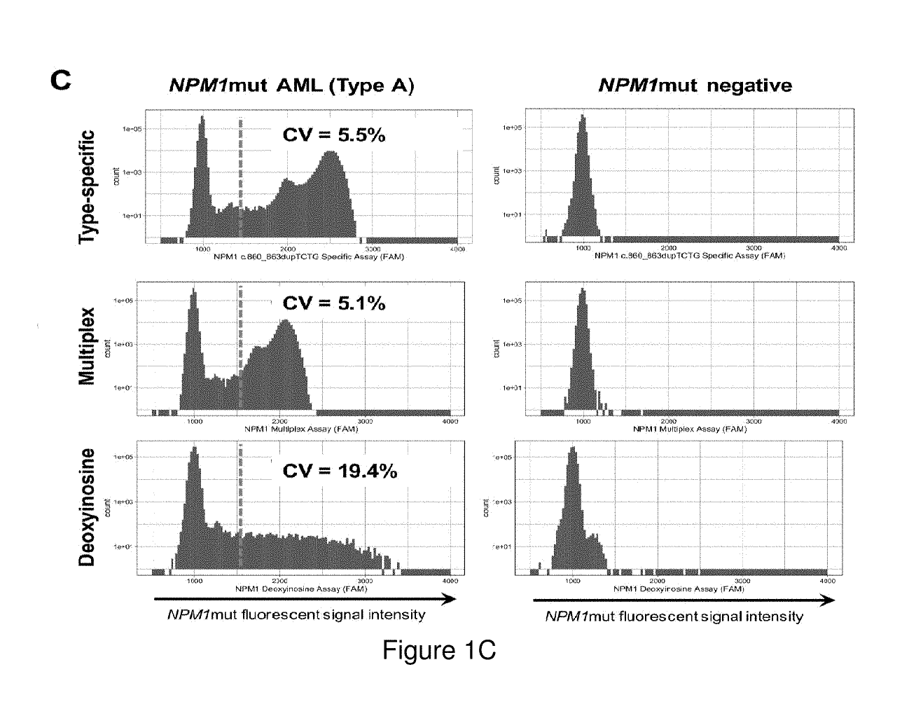

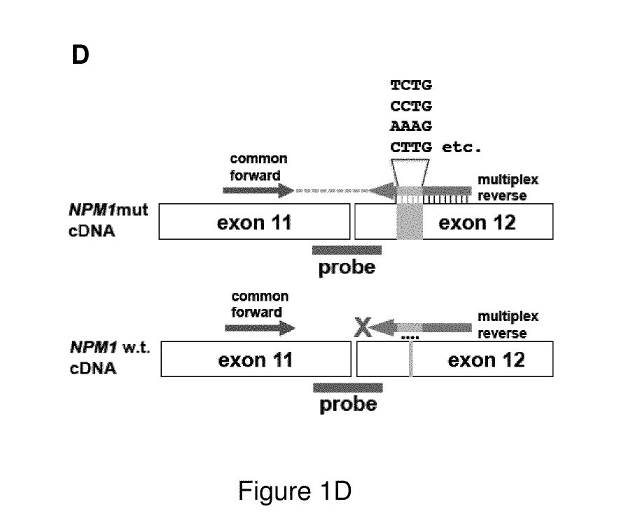

In parallel, assays were synthesized in which the common insertion site was spanned by universal bases deoxyinosine (dI) (Watkins and SantaLucia, Nucleic Acids Res., (2005); 33(19):6258-67) and 5-nitroindole (5NI) (Loakes et al., Nucleic Acids Res, (1995); 23(13):2361-6), which have relaxed base-pairing properties relative to the four standard nucleotides permitting base-pairing across a range of insertion sequences. As a baseline, the established EAC RQ-PCR-based assay for NPM1 type A mutations (Gorello et al. 2006) was adapted to dPCR. The performance of these assays was compared using cDNA from an AML patient confirmed positive for a NPM1 type A mutation by clinical sequencing (Genoptix). As shown in the histogram in FIG. 1C both the EAC type-specific assay and the multiplex assay produced distinct positive droplets indicative of successful amplification of mutated NPM1 with coefficients of variation (CV) of 5.5% and 5.1%, respectively, among positive droplets. In contrast, dPCR reactions conducted with dI-containing primers produced a non-distinct smear of positive droplets (CV=19.4%; FIG. 1C), rendering it less useful for MRD detection. 5NI-containing primers failed to produce amplified product. Given that the multiplex assay was both the most successful and cost-effective option tested, the multiplex assay was evaluated on other NPM1 mutations and in serial AML cases.

### Example 3: Agreement with Established Assays

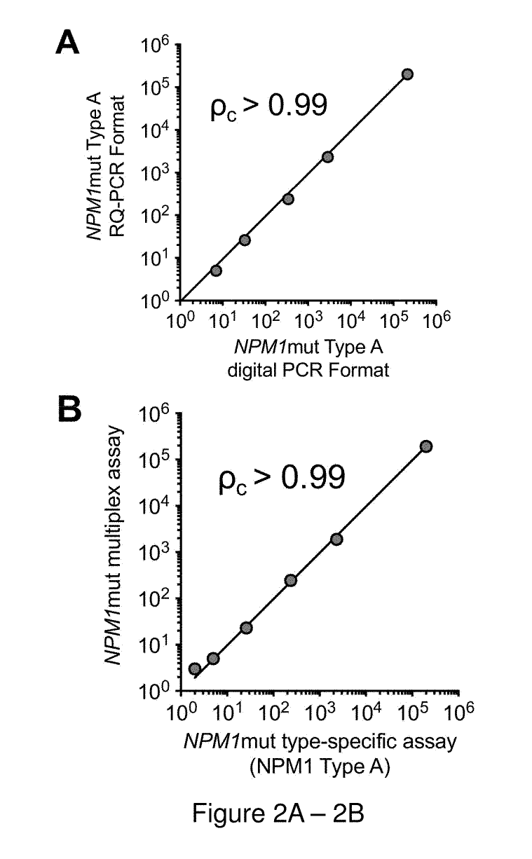

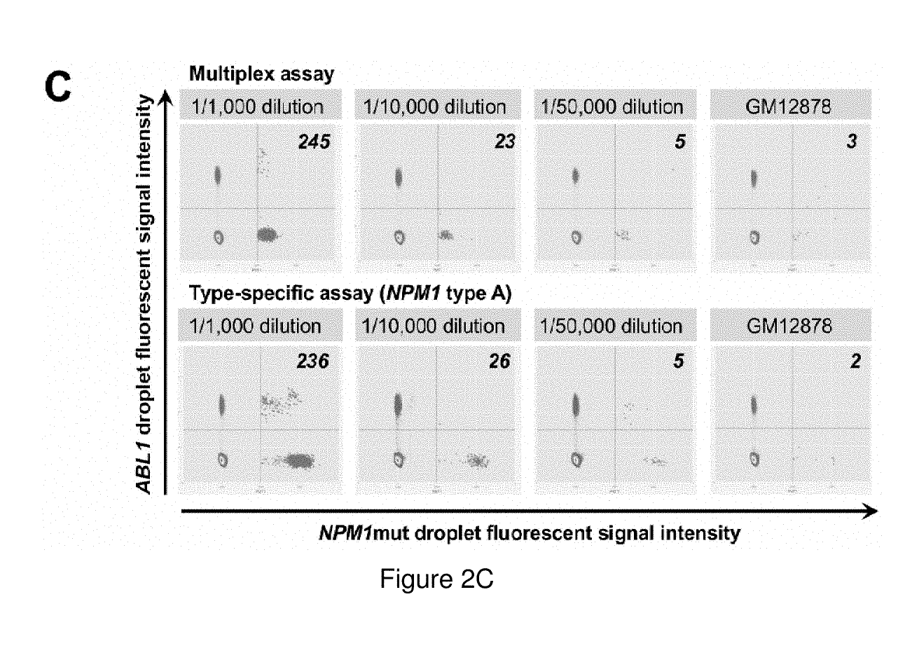

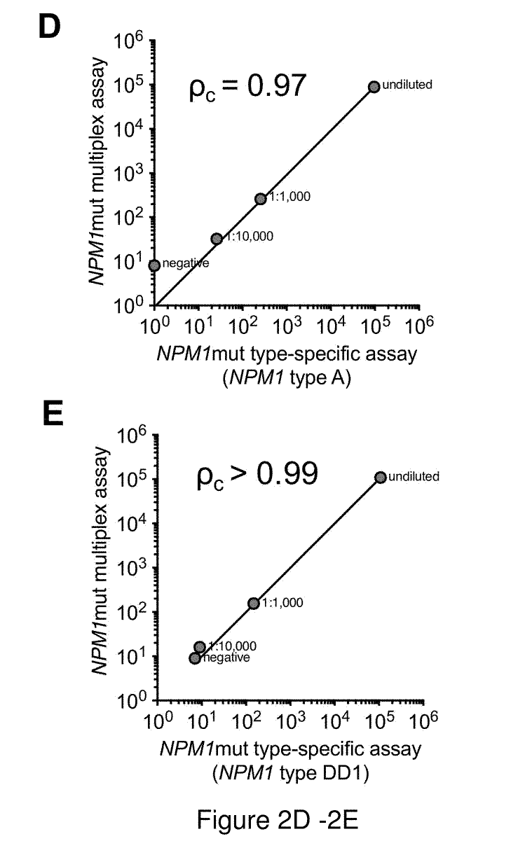

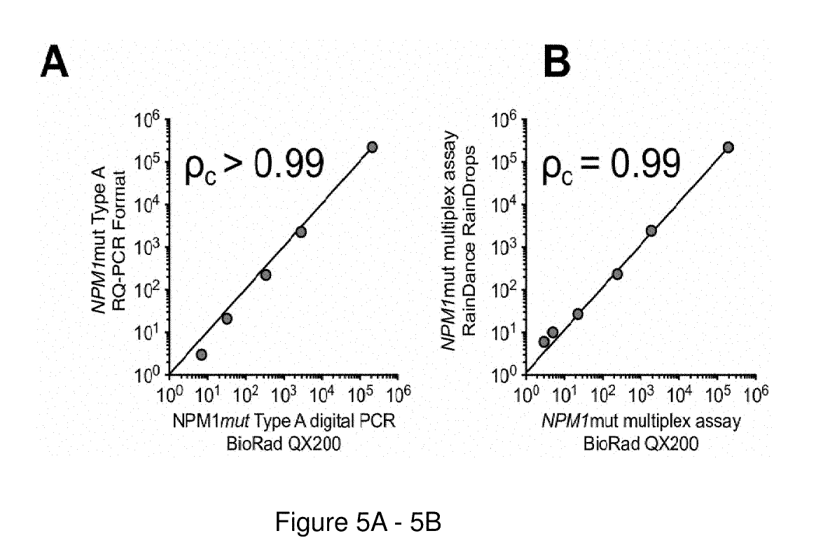

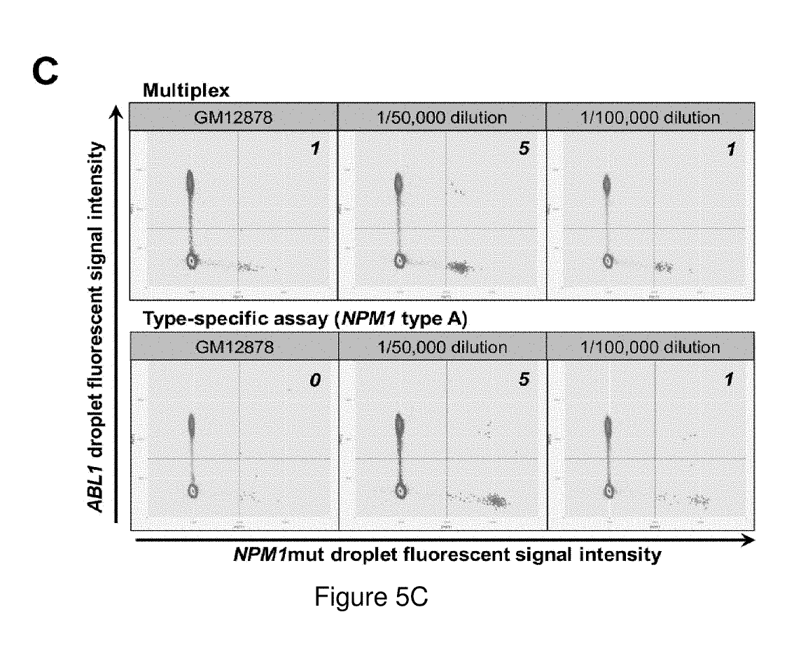

To test whether the NPM1 multiplex assay demonstrates agreement with established subtype-specific RQ-PCR and dPCR assays already used in MRD assessment, a spike-in dilution series was created of the cell line OCI-AML3 (NPM1 type A) into GM12878 (NPM1 wild type) ranging from 1:1,000 cells to 1:50,000 cells (OCI-AML3:GM12878). NPM1 mutation subtype and variant allele fraction (VAF) were verified by next generation sequencing (NGS). For each dilution, NPM1 mutant transcript copies per 10⁴ ABL1 transcript copies (NPM1mut/10⁴ ABL1) were quantified according methods and reporting convention established by Gorello et al. (2006) The established type A allele-specific assays in RQ-PCR were then compared to both type-specific and multiplex assays in dPCR. For each comparison, Lin's concordance correlation coefficient (ρ_{c}), which evaluates the extent to which pairwise comparisons agree (i.e. fit to a diagonal line with a slope of 1 and y-intercept of 0), was computed. Previous studies noted general concordance between MRD measurements obtained by RQ-PCR and dPCR using allele-specific primers across a range of both common and rare NPM1 mutations (Bacher et al, Br J Haematol, (2014); 167(5):710-4) Indeed, excellent concordance was observed between RQ-PCR and dPCR across the range of cell dilutions tested (p_{c}=0.998; 95% c.i. 0.994-0.999; RQ-PCR vs. dPCR) (FIG. 2A). NPM1mut/10⁴ ABL1 ratios were further concordant when comparing the new NPM1 multiplex assay to the EAC type-specific assay in dPCR (p_{c}=0.999; 95% c.i. 0.997-0.999; multiplex vs EAC) (FIG. 2B), producing essentially identical NPM1mut/10⁴ ABL1 ratios (FIG. 2C). The 1:50,000 dilution demonstrated NPM1mut/10⁴ ABL1 ratios just over under double the background seen in negative control GM12878. However, increasing cDNA input 10-fold improved signal-to-noise for 1:50,000 cell dilution (FIG. 5C). This concordance was preserved when using an alternative dPCR technology (BioRad QX200; FIGS. 5A and 5B) indicating that results across platforms are generally comparable.

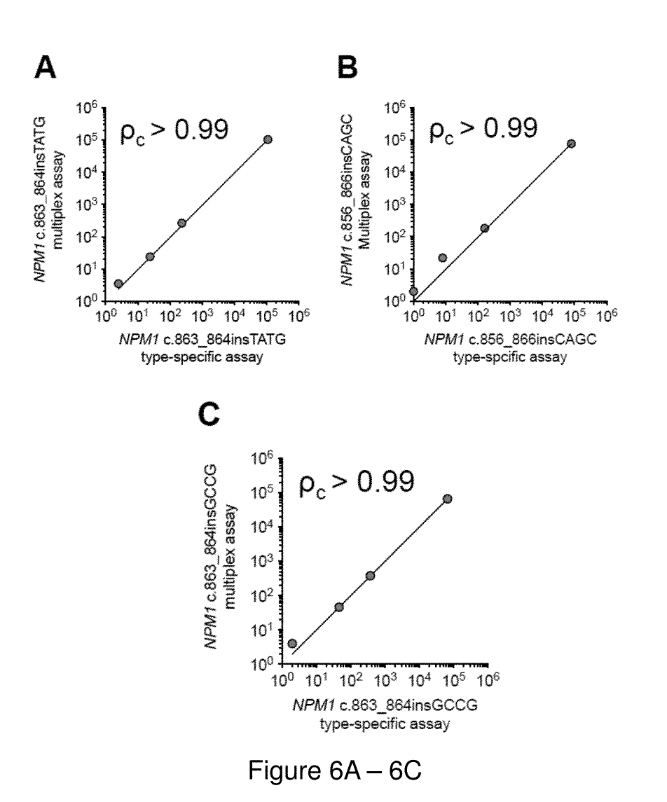

To transition from cell lines to primary samples, the NPM1 multiplex assay was further tested against type-specific assays in primary AML samples identified as positive for either the common NPM1 type A mutation (FIG. 2D) or the rare type DD1 (c.863_864insCTTG) (FIG. 2E), c.863_864insTATG, c.865_866insCAGC, and c.863_864insGCCG (FIGS. 6A-6C) diluting cDNA from primary samples into healthy cord blood (CB) cDNA at 1:1,000 and 1:10,000 (w/w). Concordance between the type-specific and multiplex assay was noted for both the type A (p_{c}=0.995; 95% c.i. 0.982-0.999) and type DD1 (p_{c}=1.000; 95% c.i. 1.000-1.000) subtypes in primary AML samples.

To determine the lower limit of detection of multiplex assay, background signal was measured in a panel of 12 NPM1mut negative samples. The panel included primary AML samples (bearing rearrangements rarely found to co-occur with NPM1 such as CBFI3-MYH11 or AML1/ETO), bone marrow from healthy donor, cord blood as well as the GM12878 and MV411 cell lines. Total NPM1 transcripts were also quantified in the same set of samples using an NPM1-universal primer with binding site downstream of the 863 position in exon 12 of NPM1 transcript. NPM1-universal primer non-selectively amplifies all NPM1 transcripts irrespective of the presence or absence of a 4 nt insertion (i.e. the number of NPM1 copies counted consist of wild-type and, if present, mutant transcripts). Background levels in 12 NPM1mut negative primary samples and cell lines tested ranged from 0-7 NPM1mut/10⁴ ABL1 (median: 1 NPM1mut/10⁴ ABL1) for the multiplex assay compared to 0-1 NPM1mut (median: 0 NPM1mut/10⁴ ABL1) for the type-specific assay (NPM1mut type A). Thus, the limit of detection (LOD) for the multiplex and type A-specific assays under dPCR conditions are estimated at 2.4 and 1.0, respectively, for normal primary blood samples; well below reported decision cutoffs of 100-1000 NPM1mut/l10⁴ ABL1 copies. Both allele-specific and massively multiplex assays produced a single amplicon as determined by gel electrophoresis thus further corroborating specificity.

The multiplex dPCR assay for mutated NPM1 demonstrated overall agreement with established type-specific assays in both the spike-in dilution series and in primary AML cells for both common and rare NPM1 subtypes. Moreover, NPM1mut/10⁴ ABL1 ratios produced by the multiplex assay agreed with established EAC assays.

### Example 4: Other Rare NPM1 Mutation Types

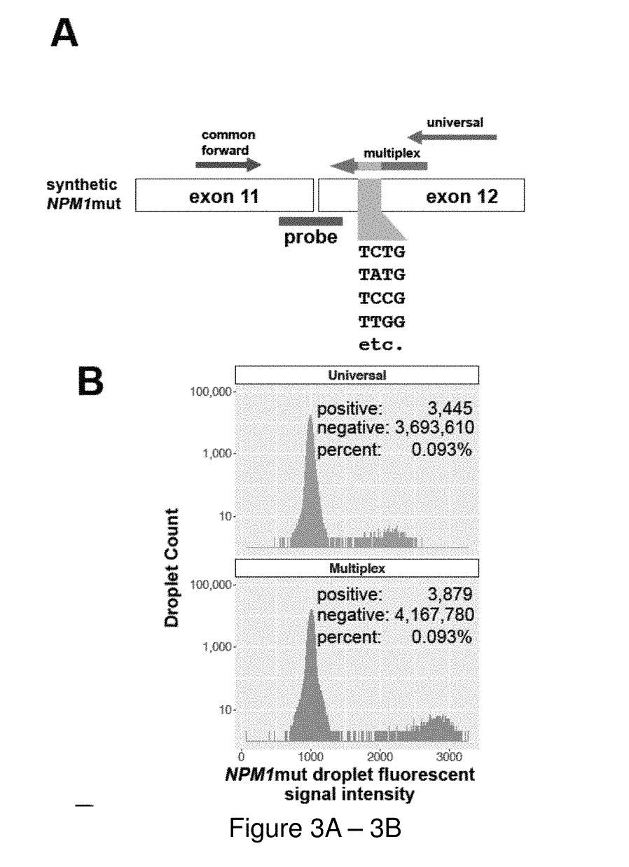

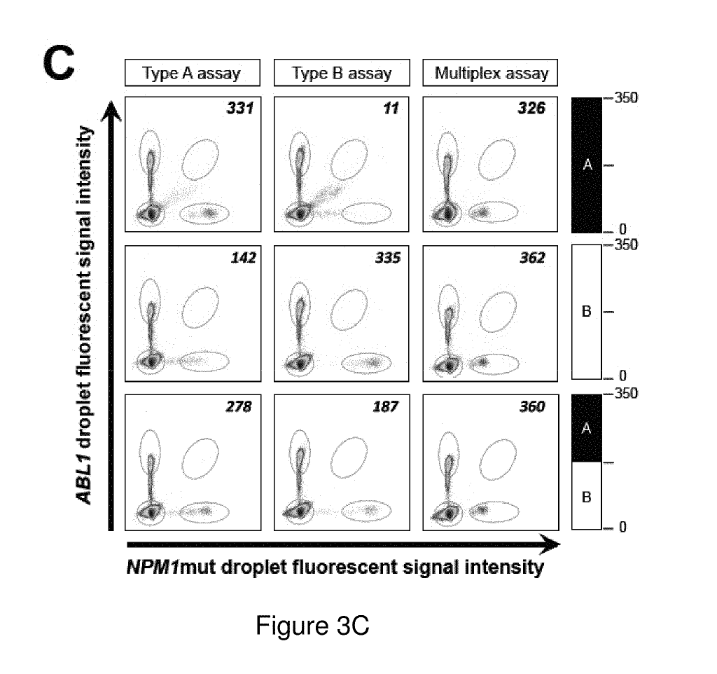

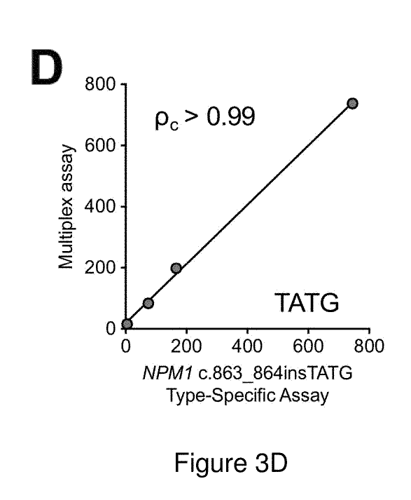

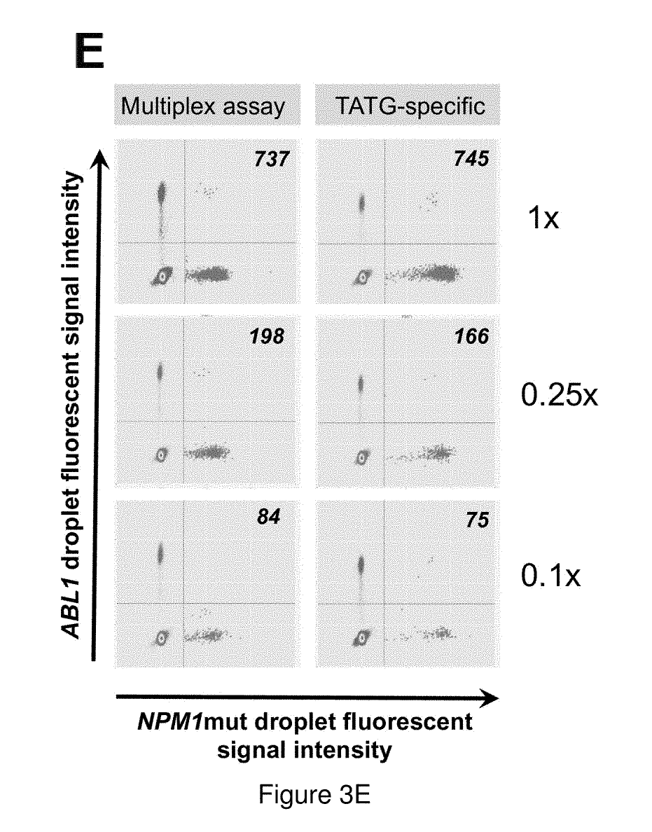

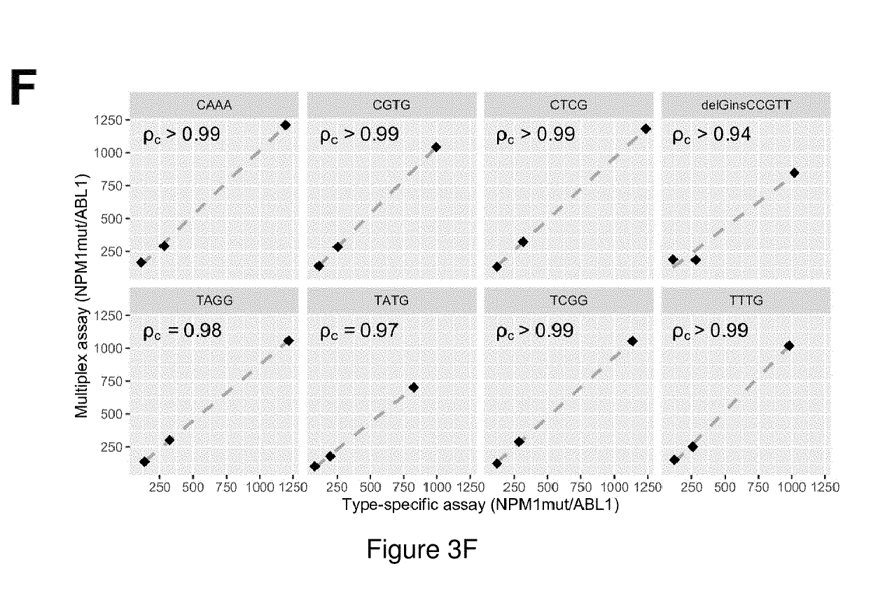

To assess detection of rare NPM1 mutations, synthetic NPM1-mutated templates with rare insertion sequences reported in Ivey et al. (N Engl J Med, (2016); 374(5):422-33) were synthesized along with the published type-specific assay for those mutations. NPM1 copies were determined using both the NPM1 multiplex assay and type-specific assay, testing a subset of rare NPM1 insertions on dual dPCR platforms. First, NPM1 c.863_864insTATG synthetic targets in a dilution series were spiked into GM12878 alongside unspiked GM12878 controls. NPM1mut/10⁴ ABL1 ratios obtained by type-specific and multiplex assay were concordant (ρ_{c}=0.998; 95% c.i. 0.981-1.000) (FIG. 3A) producing both similar values as well as distinct clusters of positive dPCR partitions/droplets (FIG. 3B). Measurements of NPM1 mutation copies were also consistent across dPCR platforms using the NPM1 multiplex assay (ρ_{c}=0.985; 95% c.i. 0.861-0.999) (FIG. 3C and Table 3). Synthetic NPM1 mutation spike-in dilution series were created for 8 rare NPM1 mutations and compared NPM1mut/10⁴ ABL1 ratios derived from both multiplex and type-specific assays (FIG. 3D). All of the NPM1 mutations demonstrated substantial concordance between type-specific and multiplex assays (ρ_{c}=0.97-0.99) with the notable exception of NPM1 c.864_865delGinsCCGTT, which contains an extra mismatch likely leading to reduced PCR priming efficiency. The multiplex assay was responsive to MRD in all cases tested.

TABLE 3
Detection of rare NPM1 mutations using multiplex
or type- specific assays on dPCR platforms

| | RainDance RainDrops | | BioRad QX200 | |
|---|---|---|---|---|
| Insertion | Multiplex | Type-specific | Multiplex | Type-specific |
| CAAA | 1035 | 1011 | 1210 | 1173 |
| CGTG | 819 | 965 | 1043 | 1005 |
| TATG | 745 | 745 | 702 | 813 |
| TCGG | 950 | 1039 | 1054 | 1099 |
| TTTG | 853 | 946 | 1019 | 972 |

### Example 5: Monitoring of MRD in Serial AML Cases

To ascertain the potential of the multiplex assay in patient care, molecular MRD levels were determined sequentially in two patients diagnosed with AML according to the WHO classification under care of the Leukemia Program of Weill Cornell-New York Presbyterian Hospital. Patients were additionally required to have mutated NPM1 confirmed by an independent diagnostic laboratory. Bone marrow aspirates (BMA) and/or peripheral blood (PB) were collected periodically from AML patients seen during the course of their care. One of these patients presented to our clinic without prior sequencing having been diagnosed for NPM1-mutated AML only by capillary electrophoresis.

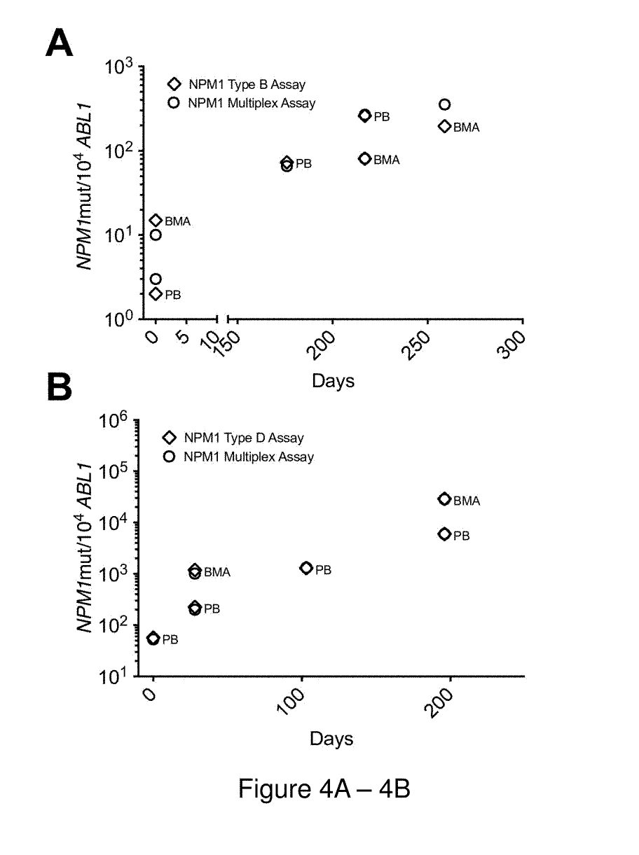

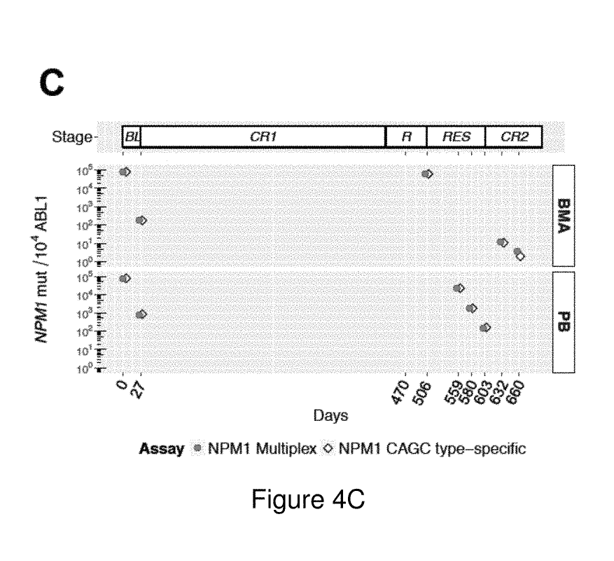

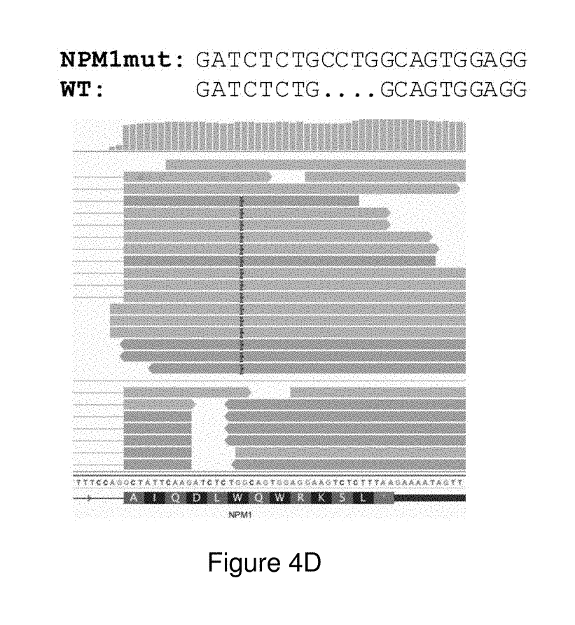

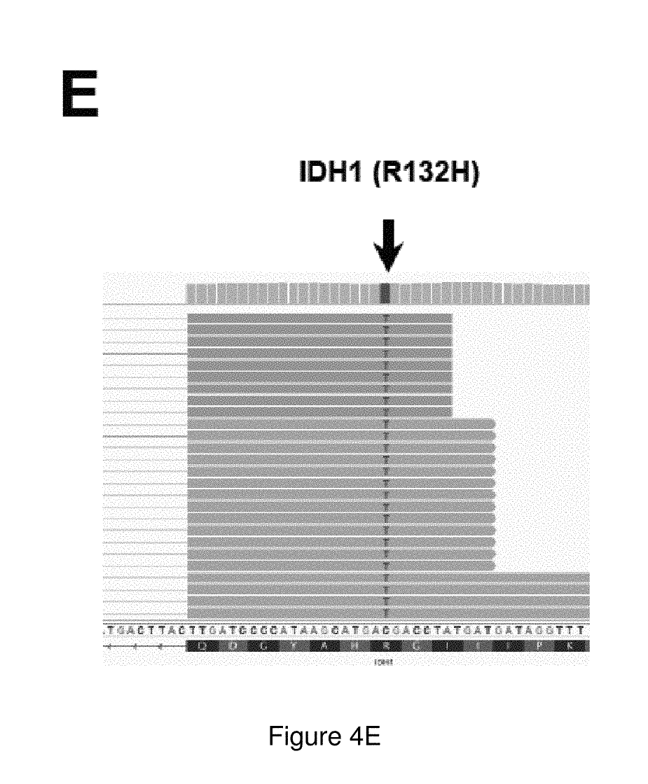

Patient 1 was confirmed positive for NPM1 type B mutation (c.863_864insCATG) by routine clinical sequencing performed as standard care (Genoptix). NPM1 MRD levels were assessed over a period of 259 days (FIG. 4A). Peripheral blood (PB) and bone marrow aspirates (BMA) demonstrated low levels of NPM1 mutation (<100 NPM1mut/10⁴ ABL1) during the first 200 days as determined by both the type-specific assay (type B) and NPM1 multiplex assay, with both assays demonstrating overall agreement and trending upwards over time. NPM1 mutation levels are expected to be higher in BMA vs. PB based on previous reports (Ivey et al. 2016; Thiede et al., Blood, (2006); 107(10):4011-20). However, the present case demonstrated higher PB mutant NPM1 percentages compared to BMA at 217, 514 and 731. The patient eventually evolved to relapsed disease after 836 days.

Patient 2 is a 62 year-old woman with a history of breast cancer treated with multi-agent chemotherapy and radiation, who presented for care at our center after having been diagnosed with AML with normal cytogenetics and treated elsewhere. She received a standard cytarabine and anthracycline-based induction, followed by four cycles of high-dose cytarabine consolidation. Patient 2 entered our study with the NPM1 mutation having been diagnosed without sequencing using only capillary electrophoresis (Quest Diagnostics). A diagnostic specimen was not available to determine the patient's NPM1 sequence. The inventors thus followed NPM1 mutant MRD levels over the course of 196 days (FIG. 4B) using the NPM1 multiplex assay. A surge in mutated NPM1 transcripts was noted in BM from day 28 reaching 1,012 NPM1mut/10⁴ ABL1. Leftover RNA initially isolated for MRD assessment was submitted for ultra-deep targeted mRNA-seq hybrid capture sequencing to determine the NPM1 subtype. This analysis revealed the NPM1 subtype as type D (c.863_864insCCTG) supported by 48/14,271 reads (0.34%) (FIG. 4D) and incidentally identified an IDH1 (p.Arg132His; R132H) unknown in this patient supported by 32/3,679 reads (0.9%) (FIG. 4E). The type-specific and multiplex assay closely tracked each other with levels of mutant NPM1 gradually rising in the PB and BMA during the course of MRD monitoring through this patient's eventual relapse 6 months later. Upon relapse, clinical DNA sequencing (Genoptix) identified the IDH1 R132H mutation at 6% VAF but not the NPM1 type D mutation. Targeted deep DNA sequencing then confirmed the presence of the NPM1 type D mutation at 3% VAF. The patient is currently undergoing treatment with an investigational IDH1 inhibitor, which was associated with an observed reduction in mutated NPM1 transcripts during the resistant disease stage.

Patient 3 presented a rare NPM1 mutation subtype (c.865_866insCAGC) confirmed by routine clinical sequencing. A type-specific assay was designed to retrospectively monitor NPM1 status alongside multiplex assay over a period of 660 days (FIG. 4C). NPM1mut copies drastically decreased after standard induction therapy after day 27 consistent with treatment response and progression into remission status. The patient received a stem cell transplant and remained in remission for more than 1 year, after which the patient relapsed as shown by the increase in NPM1mut copies with levels comparable to those observed at baseline. Re-emergence of an IDH1 mutation present at baseline was observed by NGS and at this point treatment was started with an IDH1 inhibitor under clinical trial. Finally, the patient evolved to resistant disease and secondary remission with NPM1mut levels trending downwards. The patient is currently in second remission. Type-specific and multiplex assays showed close agreement at all times in a case that is not otherwise tractable for clinically reportable molecular MRD evaluation by the most common RQ-PCR tests.

### Example 6: Cross-Detection Between Existing RQ-PCR-Based NPM1 Allele-Specific Assays

Allele-specific NPM1 assays are able to cross-detect other NPM1 subtypes, but not wild type NPM1. Representative RQ-PCR amplification curves (FIG. 1A) demonstrate the ability of the established Europe Against Cancer (EAC) NPM1 type A assay (Gorello et al. 2006) to detect 2000 copies of reference standards for NPM1 type A or type B insertions (Ipsogen) alongside wild-type NPM1. The type A assay discriminated between type A mutation and wild type NPM1. However, it was notably less selective between NPM1 type A and type B mutations; achieving a PCR amplification plateau regardless of mutation type, albeit with delayed kinetics. Cross-detection between combinations of the most common NPM1 mutations and the standard EAC assays varied substantially (FIG. 1B), but with consistently negligible affinity for wild-type NPM1. The observed specificity for NPM1 mutations vs. wild-type is thus likely conferred because mutant NPM1 primers need to loop out their insertion sequence in an energetically unfavorable manner to successfully amplify wild type NPM1 with the insertion sequence itself being a significant but lesser contributor to specificity. These observations suggested to us that cross-detection between NPM1 insertion mutations could form the basis of a robust assay. It was thus reasoned that most NPM1-mutated AMLs could be detected by a multiplex pool of assays against all known and theoretical insertion mutations.

A single test, which is specific for mutated NPM1 but simultaneously robust to the particular subtype thereby simplifying implementation of the assay in laboratories while reducing the likelihood of misdiagnoses, was designed. The multiplex assay was effective across a range of diverse common and rare NPM1 subtypes with overall concordance with validated type-specific assays. NPM1 levels are expressed by the new assay in the same units as EAC assays, thus enabling comparison to concurrent and historical studies following EAC protocols (Ivey et al. 2016; Thiede et al. 2006; Gorello et al. 2006). Uniquely, the presented test was effective in the absence of sequence information when sequentially analyzing a patient diagnosed by capillary electrophoresis and available diagnostic specimen. Deep RNA-seq of this patient during a spike in mutant NPM1 copies (˜1000 NPM1mut/10⁴ ABL1) revealed a type D mutation at low VAF (˜0.4%), which enabled us to confirm that the multiplex assay agreed with type-specific assay. An IDH1 R132H mutation was discovered during this sequencing, which was ultimately present by diagnostic sequencing 6 months later at relapse. This finding suggests an approximate NPM1 transcript copy threshold where deep sequencing might be useful for patients and requires further exploration. Overall, these data indicate that a single, easily deployed NPM1 mutation test can effectively simplify NPM1 mutation testing for laboratories while accommodating the complexities associated with NPM1 quantification.

1. A method of detecting mutant nucleic acid molecules of a gene in a sample, comprising:
providing a sample suspected of containing mutant nucleic acid molecules of said gene;
performing digital PCR on said sample using a first primer set, a second primer, and a first probe, wherein
(i) the first primer set comprises multiple primers, wherein the multiple primers anneal specifically to mutant nucleic acid molecules of the gene that each comprise an insertion of at least one nucleotide at a specific site in the gene, and the multiple primers are degenerate in at least one nucleotide position corresponding to the position of an inserted nucleotide in the mutant nucleic acid molecules;
(ii) the second primer anneals specifically to a sequence in said gene to permit generation of amplicons when used with the first primer set in the digital PCR, and
(iii) the first probe hybridizes to a sequence in said gene located between the site of insertion and the sequence to which the second primer hybridizes.

2. The method of claim 1, wherein the multiple primers in the first primer set are degenerate in 2-4 nucleotide positions.

3. The method of claim 1, wherein said multiple primers in the first primer set comprises nucleotides at four adjacent positions (NNNN) corresponding to positions of inserted nucleotides in the mutant nucleic acid molecules, wherein N at each position is independently A, C, G, T, dI or 5NI, and wherein the multiple primers are degenerate at at least two of the four positions.

4. The method of claim 3, wherein the multiple primers are degenerate at all four positions.

5. The method according to claim 1, wherein degeneracy at a nucleotide position in the first primer set is achieved by using a mixture of selected nucleotides at a predetermined ratio.

6. The method of claim 5, wherein the mixture of selected nucleotides are a mixture of A, C, G, and T, a mixture of C and T, a mixture of A and T, or a mixture of A and G.

7. The method of according to claim 1, wherein the first primer set and the second primer are designed to provide amplicons of about 75-200 base pairs.

8. The method of according to claim 1, wherein the gene is NPM1.

9. The method of claim 8, wherein the specific site of insertion is between positions 863 and 864, positions 860 and 861, positions 861 and 862, and positions 867 and 868, of the wild-type NPM1 coding sequence.

10. The method of claim 9, wherein the specific site of insertion is between positions 863 and 864, and wherein when said second primer is a forward primer, said second primer hybridizes to a sequence in exon 9, 10, 11 or a combination of exons 9, 10 and 11 of NPM1, and when said second primer is a reverse primer, said second primer hybridizes to a sequence in exon 12 of NPM1.

11. The method of claim 8, wherein when said multiple primers in the first primer set are forward primers, said second primer is a reverse primer and each primer in the first primer set comprises the sequence, 5′-TCTGNNNNGCAGTGGAGGAAG-3′ (SEQ ID NO: 12), wherein N at each position is independently A, C, T, G, dI or 5NI, and at least one of the four positions denoted by N is degenerate; and
wherein when said multiple primers in the first primer set are reverse primers, said second primer is a forward primer and each primer in the first primer set comprises the sequence, 5′-CTTCCTCCACTGCNNNNCAGA-3′ (SEQ ID NO: 4), wherein N at each position is independently A, C, T, G, dI or 5NI, and at least one of the four positions denoted by N is degenerate.

12. The method of claim 11, wherein at least one N is fixed to a nucleotide selected from the group consisting of A, T, G or C.

13. The method of claim 12, wherein said multiple primers in the first primer set are forward primers represented by the sequence to 5′-TCTGYWTGGCAGTGGAGGAAG-3′ (SEQ ID NO: 13), wherein Y is C or T and W is A or T; or
wherein said multiple primers in the first primer set are reverse primers represented by the sequence 5′-CTTCCTCCACTGCCAWRCAGA-3′ (SEQ ID NO: 14), wherein W is A or T and R is A or G.

14. The method of claim 10, wherein the second primer comprises the sequence, 5′-GAAGAATTGCTTCCGGATGACT-3′ (SEQ ID NO: 1).

15. The method of claim 10, wherein the first probe comprises the sequence, 5′-ACCAAGAGGCTATTCAA-3′ (SEQ ID NO: 2).

16. The method of claim 1, wherein the first probe comprises a fluorescent label and a quencher.

17. The method of claim 1, wherein the mutant nucleic acids are RNA, cDNA or DNA.

18. The method of claim 10, further comprising quantifying the mutated nucleic acids of the NPM1 gene in the sample.

19. The method of claim 18, further comprising quantifying nucleic acids of a second gene in the sample and generating a ratio of mutant nucleic acids of the NPM1 gene to the nucleic acids of the second gene.

20. The method of claim 19, wherein the second gene is ABL1, wild-type NPM1, or total NPM1.

21. The method of claim 20, wherein a second probe that specifically hybridizes to ABL1 is used to quantify ABL1, and wherein the second probe comprises a second fluorescent label different from the fluorescent label of the first probe.

22. The method according to claim 18, wherein the number of any mutated NPM1 transcript greater than or equal to a detection limit is indicative of cancer or residual cancer cells.

23. The method of claim 22, where the cancer is a blood cancer.

24. The method of claim 23, wherein the blood cancer is selected from the group consisting of non-Hodgkin's lymphoma, acute promyelocytic leukemia, myelodysplastic syndrome, acute lymphocytic leukemia and acute myelogenous leukemia.

25. The method of claim 1, wherein the sample is a sample of a cancer patient who has undergone cancer therapy.

26. A kit comprising a first primer set which comprises multiple primers, wherein said multiple primers specifically anneal to mutant NPM1 nucleic acid molecules encompassing an insertion of at least one nucleotide at a specific site in NPM1, and wherein said multiple primers are degenerate in at least one position corresponding to a position of an inserted nucleotide in the mutant NPM1 nucleic acid molecules.

27. The kit of claim 26, wherein said specific site is between positions 859 and 860, positions 860 and 861, positions 861 and 862, positions 862 and 863, positions 863 and 864, positions 864 and 865, positions 865 and 866, positions 866 and 867, and positions 867 and 868, of the wild-type NPM1 coding sequence.

28. The kit of claim 26, wherein said multiple primers within the first primer set are degenerate in at least one of four adjacent positions (5′ NNNN 3′) corresponding to positions of inserted nucleotides in the mutant NPM1 nucleic acid molecules, and wherein N at each position is independently A, C, G or T.

29. The kit of claim 28, wherein at least one N is fixed to a nucleotide selected from the group consisting of A, T, G or C.

30. The kit of claim 29, wherein the first N is Y, the second N is W, the third N is T and the fourth N is G, wherein Y is C or T and W is A or T

31. The kit of claim 30, wherein said multiple primers in the first primer set are forward primers represented by the sequence to 5′-TCTGYWTGGCAGTGGAGGAAG-3′ (SEQ ID NO: 13), or said multiple primers in the first primer set are reverse primers represented by the sequence to 5′-CTTCCTCCACTGCCAWRCAGA-3′ (SEQ ID NO: 14), wherein W is A or T and R is A or G.

32. The kit of claim 26, further comprising a second primer that hybridizes to a sequence in NPM1, wherein the first primer set and the second primer are designed to provide amplicons of about 75-200 base pairs in length.

33. The kit of claim 32, further comprising a probe hybridizes to a sequence in NPM1 located between the site of insertion in NPM1 and the sequence in NPM1 to which the second primer hybridizes.

34. The kit of claim 33, wherein said probe is labeled with a fluorophore and a quencher.
